(19) United States
(12) Patent Application Publication
(10) Pub. No.: US 2021/0339044 A1
(43) Pub. Date: Nov. 4, 2021

(54) RETINAL IRRADIATION SYSTEM AND METHOD TO IMPROVE OCULAR FUNCTION AND HEALTH IN THE NORMALLY AGING EYE

(71) Applicant: Ojai Retinal Technology, LLC, Los Angeles, CA (US)

(72) Inventor: Jeffrey K. Luttrull, Los Angeles, CA (US)

(73) Assignee: Ojai Retinal Technology, LLC, Garland, TX (US)

(21) Appl. No.: 17/379,027

(22) Filed: Jul. 19, 2021

Related U.S. Application Data
(63) Continuation-in-part of application No. 16/540,886, filed on Aug. 14, 2019, now abandoned.
Application No. 16/540,886 is a continuation of application No. 16/241,812, filed on Jan. 7, 2019, now Pat. No. 10,434,330.
Application No. 16/241,812 is a continuation-in-part of application No. 16/203,970, filed on Nov. 29, 2018, now Pat. No. 10,952,899.
Application No. 16/203,970 is a continuation-in-part of application No. 15/818,216, filed on Nov. 20, 2017, now Pat. No. 10,194,798.
Application No. 15/818,216 is a continuation-in-part of application No. 17/341,666, filed on Jun. 8, 2021.
Application No. 17/341,666 is a continuation-in-part of application No. 16/996,308, filed on Aug. 18, 2020, now Pat. No. 11,033,749.
Application No. 16/996,308 is a continuation of application No. 15/918,487, filed on Mar. 12, 2018, now Pat. No. 10,874,873.
Application No. 15/918,487 is a continuation-in-part of application No. 15/460,821, filed on Mar. 16, 2017, now abandoned.
Application No. 15/460,821 is a continuation-in-part of application No. 15/214,726, filed on Jul. 20, 2016, now Pat. No. 10,531,908.
Application No. 15/214,726 is a continuation-in-part of application No. 14/922,885, filed on Oct. 26, 2015, now Pat. No. 9,427,602.
Application No. 14/922,885 is a continuation-in-part of application No. 14/607,959, filed on Jan. 28, 2015, now Pat. No. 9,168,174.
Application No. 14/607,959 is a continuation-in-part of application No. 13/798,523, filed on Mar. 13, 2013, now Pat. No. 10,219,947.
Application No. 13/798,523 is a continuation-in-part of application No. 13/481,124, filed on May 25, 2012, now Pat. No. 9,381,115.
Application No. 15/918,487 is a continuation-in-part of application No. 15/629,002, filed on Jun. 21, 2017, now Pat. No. 10,278,863.
Application No. 15/629,002 is a continuation-in-part of application No. 15/583,096, filed on May 1, 2017, now Pat. No. 10,953,241.
Application No. 15/583,096 is a continuation-in-part of application No. 15/232,320, filed on Aug. 9, 2016, now Pat. No. 9,962,291.
Application No. 15/232,320 is a continuation-in-part of application No. 15/178,842, filed on Jun. 10, 2016, now Pat. No. 9,626,445.
Application No. 15/178,842 is a continuation-in-part of application No. 14/921,890, filed on Oct. 23, 2015, now Pat. No. 9,381,116.
Application No. 14/921,890 is a continuation-in-part of application No. 14/607,959, filed on Jan. 28, 2015, now Pat. No. 9,168,174.
Application No. 14/607,959 is a continuation-in-part of application No. 13/798,523, filed on Mar. 13, 2013, now Pat. No. 10,219,947.
Application No. 13/798,523 is a continuation-in-part of application No. 13/481,124, filed on May 25, 2012, now Pat. No. 9,381,115.
(60) Provisional application No. 63/056,937, filed on Jul. 27, 2020.

(51) Int. Cl. A61N 5/06; A61B 3/10

(52) U.S. Cl. CPC A61N 5/0622; A61N 2005/0628; A61B 3/102

(57) ABSTRACT

At least one pulsed light beam is applied to eye tissue to raise the temperature of the eye tissue to photostimulate the tissue. The at least one light beam has parameters of wavelength, duty cycle, power and pulse train duration selected to achieve a therapeutic or prophylactic effect while not permanently damaging the eye tissue. The eye tissue photostimulation improves ocular health and function and slows or prevents disorders associated with a normally aging eye.

### RELATED APPLICATIONS

This application claims the benefit of U.S. Provisional Application No. 63/056,937, filed on Jul. 27, 2020. This application is also a continuation-in-part of U.S. application Ser. No. 16/540,886, filed on Aug. 14, 2019, which is a continuation of U.S. application Ser. No. 16/241,812, filed Jan. 7, 2019, which is a continuation-in-part of U.S. application Ser. No. 16/203,970, filed Nov. 29, 2018, which is a continuation-in-part of U.S. application Ser. No. 15/818,216, filed Nov. 20, 2017. This application is also a continuation-in-part of U.S. application Ser. No. 17/341,666, filed on Jun. 8, 2021, which is a continuation-in-part of U.S. application Ser. No. 16/966,308, filed on Aug. 18, 2020, which is a continuation of application Ser. No. 15/918,487, filed on Mar. 12, 2018, which is a continuation-in-part of application Ser. No. 15/460,821, filed on Mar. 16, 2017, now abandoned, which is a continuation-in-part of application Ser. No. 15/214,726, filed on Jul. 20, 2016, now U.S. Pat. No. 10,531,908, which is a continuation-in-part of application Ser. No. 14/922,885, filed on Oct. 26, 2015, now U.S. Pat. No. 9,427,602, which is a continua-tion-in-part of application Ser. No. 14/607,959, filed on Jan. 28, 2015, now U.S. Pat. No. 9,168,174, which is a continuation-in-part of application Ser. No. 13/798,523, filed on Mar. 13, 2013, now U.S. Pat. No. 10,219,947, which is a continuation-in-part of application Ser. No. 13/481,124, filed on May 25, 2012, now U.S. Pat. No. 9,381,115, said application Ser. No. 15/918,487 is a continuation-in-part of application Ser. No. 15/629,002, filed on Jun. 21, 2017, now U.S. Pat. No. 10,278,863, which is a continuation-in-part of application Ser. No. 15/583,096, filed on May 1, 2017, which is a continuation-in-part of application Ser. No. 15/232,320, filed on Aug. 9, 2016, now U.S. Pat. No. 9,962,291, which is a continuation-in-part of application Ser. No. 15/178,842, filed on Jun. 10, 2016, now U.S. Pat. No. 9,626,445, which is a continuation-in-part of application Ser. No. 14/921,890, filed on Oct. 23, 2015, now U.S. Pat. No. 9,381,116, which is a continuation-in-part of application Ser. No. 14/607,959, filed on Jan. 28, 2015, now U.S. Pat. No. 9,168,174, which is a continuation-in-part of application Ser. No. 13/798,523, filed on Mar. 13, 2013, now U.S. Pat. No. 10,219,947, which is a continuation-in-part of application Ser. No. 13/481,124, filed on May 25, 2012, now U.S. Pat. No. 9,381,115.

### FIELD OF THE INVENTION

The present invention relates to systems and processes to improve ocular function and health in the normally aging eye. In particular, the present invention utilizes application of subthreshold diode micropulse laser (SDM) beams to improve the function and health of the normally aging eye.

### BACKGROUND OF THE INVENTION

Old age, and the chronic diseases that accompany and contribute to aging, is a relatively recent phenomenon. For all but the most recent moments of the progress of human history, life spans rarely exceeded a few decades; typically cut short by trauma, starvation, exposure and/or infection. As the biological imperative is reproduction rather than an active and stimulating retirement, the body's reparative mechanisms are primarily designed to address the acute existential threats of youth. The insidious and slowly progressive degenerations of senescence are thus poor activators of these otherwise ubiquitous, highly conserved and powerful healing processes. The epidemic of aging is a modern problem.

As aging is a normal process common to all it cannot properly be considered a disease. Normal aging is accompanied by various physiologic and structural changes in the body which, while falling short of representing a disease state, may reduce function. At the same time, advancing age increases the propensity to develop a myriad of age-related disease processes. Thus, by fostering and maintaining optimal health and function in the midst of normal aging, function can be maximized and the likelihood and progression to various disease states can be minimized, thus minimizing the adverse events associated with those disease states such as vision loss or myocardial infarction.

The retinal pigment epithelium of the eye (RPE) is a monolayer of cuboidal cells that constitute a distinct and critically important layer of the eye. Embryologically, the RPE derives from neural optic epithelial cells and causes differentiation via induction of the adjacent tissues of the eye into the neurosensory retina, the vascular choroid, and the structural sclera. This process is directed by chemical mediators elaborated by the RPE during ocular development.

Embryologic defects in the RPE, if small, can result in failure of parts of the eye to develop normally. These areas are called “colobomata”. In severe cases in the absence of a normal RPE the eye cannot develop with any degree of normal structure or function. The impact of the RPE failure after normal ocular development recapitulates the inductive role as a trophic effect on adjacent tissues. Damage to or death of the RPE after birth leads to atrophy and loss of function in the neurosensory retina and choroid. Once this damage develops it tends to progress, particularly in advanced age.

In normal function, the RPE has several roles critical to visual function. These include establishment and maintenance of the blood-ocular barrier; maintenance and processing of the neurosensory retinal photoreceptor outer segments; maintenance of mass fluid flow from the vitreous through the retina into the choroidal plexus; and a unique endocrine function wherein it is responsible for maintenance of normal retinal function as a receptor and elaborator of intercellular, locally acting, and systemically acting chemical factors such as chemokines, cytokines, interleukins, and various modulators of local and systemic immunity.

The association of aging with chronic inflammation, often referred to as “inflammaging”, is now recognized as a significant component of virtually all chronic, age-related diseases, including age-related macular degeneration (AMD). Unlike acute inflammation, inflammaging is low grade, chronic, persistent, and self-perpetuating, and leads to tissue degeneration. To understand the mechanisms by which inflammaging is generated, we must first understand the essential role of the immune system in the maintenance of normal tissue function and homeostasis.

Normal cellular activity results in acquired abnormalities of protein secondary and tertiary structure (misfolding) and aggregation that, in sufficient degree, can lead to cell dysfunction. Thus, in healthy cells, there is constant surveillance and repair to maintain normal cell function and homeostasis. However, in disease these abnormalities often either escape repair and/or exceed the cell's ability to manage them successfully. The menu and severity of protein misfolding (and thus loss of normal function) is generally characteristic of the primary underlying disease process or stressor. In this case that stressor is normal aging.

Further, both normal and diseased tissues produce waste, or “self-debris”, that includes damaged cells and macromolecules. In disease, the accumulation of this waste is excessive and thus progressive, ultimately compromising tissue structure and function. At the tissue level, the mechanism employed to repair this damage and remove this waste is inflammatory-mediated (inflammation being a pre-requisite of repair) and is dependent on resident macrophages and mast cells. With aging this ‘housekeeping’ function becomes less efficient, due mainly to the combination of increased generation of self-debris and inefficient removal, requiring additional inflammatory input to maintain the tissue in a physiologic (normal) or near physiologic working state, a process that may be mediated but ultimately compromised by assembly of the “inflammasome”. The inflammasome is a multiprotein stimulus-dependent oligomer that activates the chronic inflammatory process by promoting secretion of pro-inflammatory cytokines and interleukins.

Dysregulation of inflammasomes is a feature of all chronic diseases, and may lead to inflammatory cell death termed “pyroptosis”, which is distinct from apoptosis. This heightened inflammatory state, between basal physiologic inflammation and pathologic inflammation is referred to as ‘para-inflammation’. With further aging the inflammatory stakes continue to rise, eventually escalating to require mobilization of a systemic immune response that includes the recruitment of additional leukocytes and expression of systemic pro-inflammatory cytokines. Thus, maintenance of tissue homeostasis in the aging human requires an increasing inflammatory response to address increased reparative demands that eventually moves beyond para-inflammation to a self-perpetuating and degenerative chronic inflammatory state referred to as ‘inflammaging’.

With respect to an eye, several issues are associated with normal aging and there are also disease associated pathologies located outside of the retina itself. Normal aging is associated with subtle decreases in visual function, particularly impaired dark adaptation and mesopic visual acuity. More severe compromises in these functions may be predictive of future diseases. Cataract formation is the most common cause of reversible age-related visual loss. Presbyopia is a normal aging event typically coming on at approximately thirty-five years of age, where the eye loses its ability to focus at near distances. Elevation of intraocular pressure is also a common phenomenon of aging and when severe may cause or contribute to optic nerve damage as open angle glaucoma.

Thus, there is a continuing need for systems and processes which improve ocular function and health in a normally aging eye such that vision and disorders associated with a normally aging eye are slowed or prevented. The present invention fulfills these needs, and provides other related advantages.

### SUMMARY OF THE INVENTION

The present invention generally relates to systems and processes to improve ocular function and health in a normally aging eye. Improving the ocular function and health of the eye slows or prevents disorders associated with a normally aging eye.

A system for photostimulating eye tissue of a normally aging eye comprises at least one laser console generating at least one micropulsed treatment laser light beam. The at least one treatment laser light beam has parameters to treat the retinal tissue without damaging or destroying the retinal tissue, including having a wavelength between 750 nm and 1300 nm, a duty cycle of less than 10%, and a pulse train duration of between 0.1 and 0.6 seconds. The at least one laser console may comprise a plurality of laser consoles. At least a plurality of the generated treatment laser light beams may have different wavelengths.

The at least one treatment laser light beam passes through at least one optical lens or mask to optically shape the at least one treatment laser light beam. The at least one optical lens or mask may include diffractive optics to generate a plurality of treatment light beams from the at least one treatment laser light beam which are simultaneously projected onto the retinal tissue.

A coaxial wide-field non-contact digital optical viewing camera projects the at least one treatment laser light beam, or simultaneously projects the plurality of treatment light beams, to an area of a desired site for a normally aging eye for performing retinal phototherapy or photostimulation to improve ocular health and slow or prevent normal aging disorders.

A mechanism controllably moves the at least one treatment laser light beam over substantially the entire retina, including at least a portion of the fovea. The mechanism may controllably move the at least one laser light beam during an interval between consecutive pulse applications of the at least one treatment laser light beam to a first treatment area to at least one other area of the desired site for performing retinal phototherapy or photostimulation, and subsequently returning the at least one treatment laser light beam to the first treatment area within a predetermined period of time comprising one to three milliseconds to apply another pulse application of the at least one treatment laser light beam to that first treatment area.

The tissue is preferably raised between six and eleven degrees Celsius at least during application of the at least one treatment pulsed light beam, while maintaining an average target tissue temperature over a six-minute period below or at one degree Celsius.

A process for photostimulating a normally aging eye in accordance with the present invention comprises providing a pulsed light beam having parameters of wavelength, duty cycle, power and pulse train duration selected so as to raise an eye tissue temperature to achieve a therapeutic or prophylactic effect. An average temperature rise of the eye tissue over several minutes is maintained at or below a predetermined level so as not to permanently damage the eye tissue. The pulsed light beam may have a wavelength between 530 nm and 1300 nm, a duty cycle of less than 10% and a pulse train duration of between 0.1 and 0.6 seconds. More preferably, the wavelength is between 750 nm and 1000 nm and the duty cycle is between 2% and 5%. The pulsed light beam may have a power of between 0.5 and 74 watts.

The pulsed light beam is applied to the target tissue comprising retinal tissue of the eye for less than one second to photostimulate the eye tissue without permanently damaging the eye tissue. The pulsed light beam may be applied over substantially the entire retina, including at least a portion of the fovea.

The target tissue may be raised between six and eleven degrees Celsius at least during application of the pulsed light beam, while maintaining an average target temperature over several minutes below a predetermined level. The target tissue temperature may be maintained at one degree Celsius over a six-minute period of time.

A plurality of pulsed light beams may be simultaneously applied to the target tissue. At least a plurality of the pulsed light beams may be of different wavelengths.

The light beam may be applied to a first target tissue area and between pulses of the pulsed light beam moved to one or more additional target tissue areas and then within the period of time between pulses according to the duty cycle, comprising less than one second, returned and reapplied to the first target tissue.

A first treatment to a target tissue may be performed by repeatedly applying a pulsed energy to the target tissue over a first period of time comprising less than one second so as to controllably raise a temperature of the target tissue to therapeutically treat the target tissue without destroying or damaging the target tissue and to create first level of heat shock protein activation in the target tissue. The application of the pulsed energy to the target tissue is halted for an interval of time comprising three seconds to three minute. A second treatment to the target tissue, that received the first treatment, is performed immediately after the interval of time by repeatedly reapplying the pulsed energy to the target tissue over a second period of time comprising less than one second so as to controllably raise the temperature of the target tissue to therapeutically treat the target tissue without destroying or damaging the target tissue and to create a second level of heat shock protein activation in the target tissue that is greater than the first level.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIGS. 1A and 1B are graphs illustrating the average power of a laser source compared to a source radius and pulse train duration of the laser;

FIGS. 2A and 2B are graphs illustrating the time for the temperature to decay depending upon the laser source radius and wavelength;

FIG. 3 is a diagrammatic view illustrating a system used to generate a laser light beam, in accordance with the present invention;

FIG. 4 is a diagrammatic view of optics used to generate a laser light geometric pattern, in accordance with the present invention;

FIG. 5 is a top plan view of an optical scanning mechanism, used in accordance with the present invention;

FIG. 6 is a partially exploded view of the optical scanning mechanism of FIG. 5, illustrating the various component parts thereof;

FIG. 7 illustrates controlled offsets of exposure of an exemplary geometric pattern grid of laser spots to treat the target tissue, in accordance with an embodiment of the present invention;

FIG. 8 is a diagrammatic view illustrating the use of a geometric object in the form of a line controllably scanned to treat an area of the target tissue;

FIG. 9 is a diagrammatic view similar to FIG. 8, but illustrating the geometric line or bar rotated to treat the target tissue;

FIG. 10 is a diagrammatic view illustrating an alternate embodiment of the system used to generate laser light beams for treating tissue, in accordance with the present invention;

FIG. 11 is a diagrammatic view illustrating yet another embodiment of a system used to generate laser light beams to treat tissue in accordance with the present invention;

FIGS. 12A-12D are diagrammatic views illustrated in the application of micropulsed energy to different treatment areas during a predetermined interval of time, within a single treatment session, and reapplying the energy to previously treated areas, in accordance with the present invention;

FIGS. 13-15 are graphs depicting the relationship of treatment power and time in accordance with the embodiments of the present invention;

FIGS. 16A and 16B are graphs depicting the behavior of HSP cellular system components over time following a sudden increase in temperature;

FIGS. 17A-17H are graphs depicting the behavior of HSP cellular system components in the first minute following a sudden increase in temperature;

FIGS. 18A and 18B are graphs illustrating variation in the activated concentrations of HSP and unactivated HSP in the cytoplasmic reservoir over an interval of one minute, in accordance with the present invention; and

FIG. 19 is a graph depicting the improvement ratios versus interval between treatments, in accordance with the present invention.

### DETAILED DESCRIPTION OF THE PREFERRED EMBODIMENTS

As shown in the accompanying drawings, and as more fully described herein, the present invention is directed to a system and method for delivering a pulsed energy, such as one or more light beams, or the like, having energy parameters selected to cause a thermal time-course in tissue to raise the tissue temperature over a short period of time to a sufficient level to achieve a therapeutic effect while maintaining an average tissue temperature over a prolonged period of time below a predetermined level so as to avoid permanent tissue damage. It is believed that the creation of the thermal time-course stimulates heat shock protein activation or production and facilitates protein repair without causing any damage.

The inventors have discovered that electromagnetic radiation can be applied to retinal tissue in a manner that does not destroy or damage the retinal tissue while achieving beneficial effects on eye diseases. More particularly, a laser light beam can be generated that is therapeutic, yet sublethal to retinal tissue cells and thus avoids damaging photocoagulation in the retinal tissue which provides preventative and protective treatment of the retinal tissue of the eye. It is believed that this may be due, at least in part, to the stimulation and activation of heat shock proteins and the facilitation of protein repair in the retinal tissue.

Various parameters of the light beam must be taken into account and selected so that the combination of the selected parameters achieve the therapeutic effect while not permanently damaging the tissue. These parameters include laser wavelength, radius of the laser source, average laser power, total pulse duration, and duty cycle of the pulse train.

The selection of these parameters may be determined by requiring that the Arrhenius integral for HSP activation be greater than 1 or unity. Arrhenius integrals are used for analyzing the impacts of actions on biological tissue. At the same time, the selected parameters must not permanently damage the tissue. Thus, the Arrhenius integral for damage may also be used, wherein the solved Arrhenius integral is less than 1 or unity.

Alternatively, the FDA/FCC constraints on energy deposition per unit gram of tissue and temperature rise as measured over periods of minutes be satisfied so as to avoid permanent tissue damage. Generally speaking, tissue temperature rises of between 6° C. and 11° C. can create therapeutic effect, such as by activating heat shock proteins, whereas maintaining the average tissue temperature over a prolonged period of time, such as over several minutes, such as six minutes, below a predetermined temperature, such as 1° C. or less in certain circumstances, will not permanently damage the tissue.

The inventors have discovered that generating a subthreshold, sublethal micropulse laser light beam which has a wavelength greater than 532 nm and a duty cycle of less than 10% at a predetermined intensity or power and a predetermined pulse length or exposure time creates desirable retinal photostimulation without any visible burn areas or tissue destruction. More particularly, a laser light beam having a wavelength of between 550 nm-1300 nm, and in a particularly preferred embodiment between 750 nm and 1000 nm, having a duty cycle of approximately 2.5%-5% and a predetermined intensity or power (such as between 100-590 watts per square centimeter at the retina or approximately 1 watt per laser spot for each treatment spot at the retina) and a predetermined pulse length or exposure time (such as between 100 and 600 milliseconds or less) creates a sublethal, “true subthreshold” retinal photostimulation in which all areas of the retinal pigment epithelium exposed to the laser irradiation are preserved and available to contribute therapeutically. In other words, the inventors have found that raising the retinal tissue at least up to a therapeutic level but below a cellular or tissue lethal level recreates the benefit of the halo effect of the prior art methods without destroying, burning or otherwise damaging the retinal tissue. This is referred to herein as subthreshold diode micropulse laser treatment (SDM).

SDM does not produce laser-induced retinal damage (photocoagulation), and has no known adverse treatment effect, and has been found to be an effective treatment in a number of retinal disorders (including diabetic macular edema (DME) proliferative diabetic retinopathy (PDR), macular edema due to branch retinal vein occlusion (BRVO), central serous chorioretinopathy (CSR), reversal of drug tolerance, and prophylactic treatment of progressive degenerative retinopathies such as dry age-related macular degeneration, Stargardts' disease, cone dystrophies, and retinitis pigmentosa). The safety of SDM is such that it may be used transfoveally in eyes with 20/20 visual acuity to reduce the risk of visual loss due to early fovea-involving DME.

A mechanism through which SDM is believed to work is the generation or activation of heat shock proteins (HSPs). Despite a near infinite variety of possible cellular abnormalities, cells of all types share a common and highly conserved mechanism of repair: heat shock proteins (HSPs). HSPs are elicited almost immediately, in seconds to minutes, by almost any type of cell stress or injury. In the absence of lethal cell injury, HSPs are extremely effective at repairing and returning the viable cell toward a more normal functional state. Although HSPs are transient, generally peaking in hours and persisting for a few days, their effects may be long lasting. HSPs reduce inflammation, a common factor in many disorders.

Laser treatment can induce HSP production or activation and alter cytokine expression. The more sudden and severe the non-lethal cellular stress (such as laser irradiation), the more rapid and robust HSP activation. Thus, a burst of repetitive low temperature thermal spikes at a very steep rate of change (about 7° C. elevation with each 100 μs micropulse, or 70,000° C./sec) produced by each SDM exposure is especially effective in stimulating activation of HSPs, particularly compared to non-lethal exposure to subthreshold treatment with continuous wave lasers, which can duplicate only the low average tissue temperature rise.

In an SDM treatment, a short train of micropulses typically lasting a fraction of a second irradiates a spot on the retina. It is believed that the momentary thermal shock caused by this irradiation activates dormant cytoplasmic heat shock proteins (HSPs). These activated HSPs then initiate a chain of reactions, the net result of which is the refolding or destruction of damaged cellular proteins. Whereas the initial thermal shock dies out in seconds, the actual repair work resulting from the extensive reaction chain persists for minutes and hours.

At subsecond times scales, it is believed that one of the effects of the irradiation is to activate HSPs that are present in the cytoplasm but that are dormant (inactive) because they are bound to molecules called heat shock (transcription) factors (HSF's). The irradiation separates a bound HSP complex (HSP.HSF) into free HSP and HSF molecules. The free HSF molecules combine with each other to form trimers HSF3 which in turn combine with elements of the cell's DNA called heat shock elements (HSE's) to initiate the formation of messenger RNA molecules (mRNA) that result in the formation of new HSP's. However, this production of new HSP's takes time, so the initial impact of the subsecond irradiation can be described as simply modifying the concentrations of the cell's HSP and HSF through its effect on the concentration of HSP.HSF.

The laser-induced temperature rise—and therefore the activation Arrhenius integral—depends on both the treatment parameters (e.g., laser power, duty cycle, total train duration) and on the RPE properties (e.g., absorption coefficients, density of HSPs). It has been found clinically that effective SDM treatment is obtained when the Arrhenius integrals is of the order of unity. Interestingly, the healing effect of SDM irradiation does not appear to be related to its effect on changing the initial concentration of HSPs, but is related to the conformationally-induced improvements in the reaction rate constants. From numerical simulations, the inventors have found that the Rybinski rate constant that is most important in determining the final value of undamaged proteins P is K₁₀, the right constant describing the rate at which HSPs combined with damaged proteins S repair the damaged proteins to return them to an undamaged state p.

The Rybinski et al. (2013) equations indicate that the most important SDM-affected reaction constant is K₁₀, the rate constant describing how rapidly an HSP molecule bound to a damaged protein repairs it. The equations also indicate that SDM-induced repair is much larger for an unhealthy cell than it is for a healthy cell. For example, for the unhealthy cell initially with only 45% of its proteins undamaged, the SDM-induced repair was almost 40%, compared to an improvement of a little over 6% for a healthy cell with 88% of its proteins initially undamaged. It is believed that the SDM-induced improvement in the number of undamaged proteins in a cell is due to changes in the rate constants resulting from heat-shock induced conformation changes in the HSPs.

Laser wavelengths below 550 nm produce increasingly cytotoxic photochemical effects. While laser wavelengths as low as 532 nm can be used in connection with the present invention, the lower range of these wavelengths produce increasingly cytotoxic photochemical effects and the safety margin for using such lower wavelengths is considerably smaller. For example, the safety margin for a 577 nm wavelength pulse laser is only 0.20 watts as compared to 1.92 watts for an 810 nm wavelength. Thus, preferably the present invention utilizes higher wavelengths, such as in the 750-1000 nm range and more preferably approximately 810 nm. At 810 nm, SDM produces photothermal, rather than photochemical, cellular stress. Thus, SDM is able to affect the tissue without damaging it. The clinical benefits of SDM are thus primarily produced by sub-morbid photothermal cellular HSP activation. In dysfunctional cells, HSP stimulation by SDM results in normalized cytokine expression, and consequently improved structure and function. The therapeutic effects of this “low-intensity” laser/tissue interaction are then amplified by “high-density” laser application, recruiting all the dysfunctional cells in the targeted tissue area by densely/confluently treating a large tissue area, including all areas of pathology, thereby maximizing the treatment effect. These principles define the treatment strategy of SDM described herein.

Because normally functioning cells are not in need of repair, HSP stimulation in normal cells would tend to have no notable clinical effect. The “patho-selectivity” of near infrared laser effects, such as SDM, affecting sick cells but not affecting normal ones, on various cell types is consistent with clinical observations of SDM. SDM has been reported to have a clinically broad therapeutic range, unique among retinal laser modalities, consistent with American National Standards Institute “Maximum Permissible Exposure” predictions. While SDM may cause direct photothermal effects such as entropic protein unfolding and disaggregation, SDM appears optimized for clinically safe and effective stimulation of HSP-mediated repair.

As noted above, while SDM stimulation of HSPs is non-specific with regard to the disease process, the result of HSP mediated repair is by its nature specific to the state of the dysfunction. HSPs tend to fix what is wrong, whatever that might be. Thus, the observed effectiveness of SDM in retinal conditions as widely disparate as BRVO, DME, PDR, CSR, age-related and genetic retinopathies, and drug-tolerant NAMD. Conceptually, this facility can be considered a sort of “Reset to Default” mode of SDM action. For the wide range of disorders in which cellular function is critical, SDM normalizes cellular function by triggering a “reset” (to the “factory default settings”) via HSP-mediated cellular repair.

The inventors have found that SDM treatment of patients suffering from age-related macular degeneration (AMD) can slow the progress or even stop the progression of AMD. Most of the patients have seen significant improvement in dynamic functional log MAR mesoptic visual acuity and mesoptic contrast visual acuity after the SDM treatment. It is believed that SDM works by targeting, preserving, and “normalizing” (moving toward normal) function of the retinal pigment epithelium (RPE).

SDM has also been shown to stop or reverse the manifestations of the diabetic retinopathy disease state without treatment-associated damage or adverse effects, despite the persistence of systemic diabetes mellitus. On this basis it is hypothesized that SDM might work by inducing a return to more normal cell function and cytokine expression in diabetes-affected RPE cells, analogous to hitting the “reset” button of an electronic device to restore the factory default settings. Based on the above information and studies, SDM treatment may directly affect cytokine expression via heat shock protein (HSP) activation in the targeted tissue.

Thus, the Inventors have shown that chronic progressive retinal diseases, neurodegenerative disorders associated with aging, can be prevented or slowed by irradiative treatment directed at the RPE to improve RPE function. These include the main causes of irreversible visual loss such as age-related macular degeneration (AMD), diabetic retinopathy (DR), inherited retinopathies (IRD), and open angle glaucoma (OAG).

Thus, application of SDM to retinal eye tissue, and more particularly retinal tissue, in accordance with the present invention improves ocular function and health so that disorders associated with the normally aging eye are slowed or prevented.

As indicated above, subthreshold diode micropulse laser (SDM) photostimulation has been effective in stimulating direct repair of slightly misfolded proteins in eye tissue. Besides HSP activation, another way this may occur is because the spikes in temperature caused by the micropulses in the form of a thermal time-course allows diffusion of water inside proteins, and this allows breakage of the peptide-peptide hydrogen bonds that prevent the protein from returning to its native state. The diffusion of water into proteins results in an increase in the number of restraining hydrogen bonds by a factor on the order of a thousand.

As explained above, the energy source to be applied to the target tissue will have energy and operating parameters which must be determined and selected so as to achieve the therapeutic effect while not permanently damaging the tissue. Using a light beam energy source, such as a laser light beam, as an example, the laser wavelength, duty cycle and total pulse train duration parameters must be taken into account. Other parameters which can be considered include the radius of the laser source as well as the average laser power. Adjusting or selecting one of these parameters can have an effect on at least one other parameter.

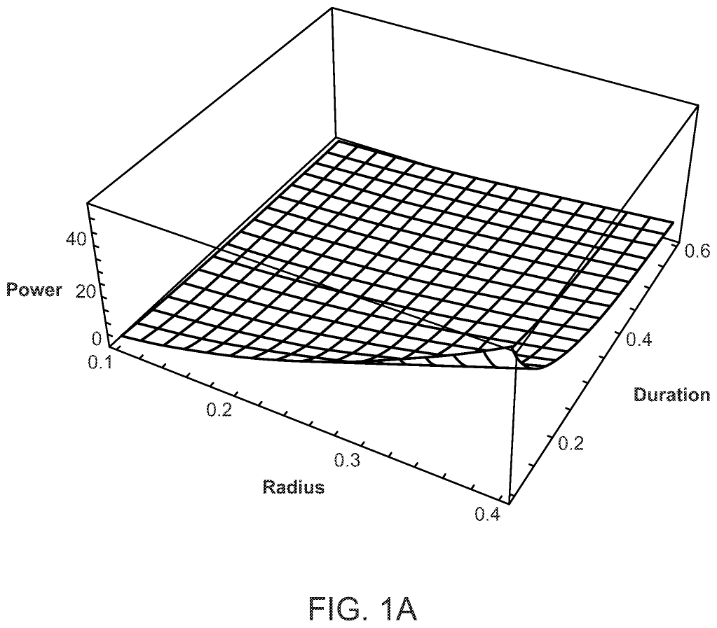

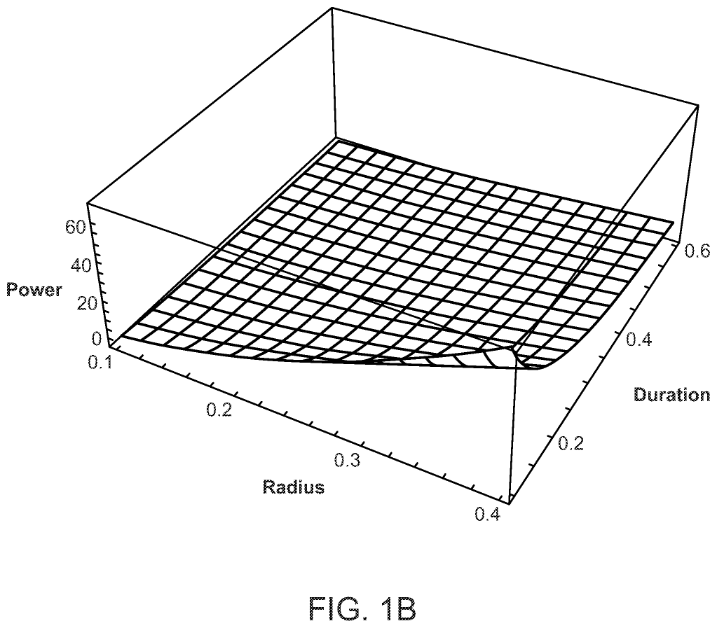

FIGS. 1A and 1B illustrate graphs showing the average power in watts as compared to the laser source radius (between 0.1 cm and 0.4 cm) and pulse train duration (between 0.1 and 0.6 seconds). FIG. 1A shows a wavelength of 880 nm, whereas FIG. 1B has a wavelength of 1000 nm. It can be seen in these figures that the required power decreases monotonically as the radius of the source decreases, as the total train duration increases, and as the wavelength decreases. The preferred parameters for the radius of the laser source is 1 mm-4 mm. For a wavelength of 880 nm, the minimum value of power is 0.55 watts, with a radius of the laser source being 1 mm, and the total pulse train duration being 600 milliseconds. The maximum value of power for the 880 nm wavelength is 52.6 watts when the laser source radius is 4 mm and the total pulse drain duration is 100 milliseconds. However, when selecting a laser having a wavelength of 1000 nm, the minimum power value is 0.77 watts with a laser source radius of 1 mm and a total pulse train duration of 600 milliseconds, and a maximum power value of 73.6 watts when the laser source radius is 4 mm and the total pulse duration is 100 milliseconds. The corresponding peak powers, during an individual pulse, are obtained from the average powers by dividing by the duty cycle.

The volume of the tissue region to be heated is determined by the wavelength, the absorption length in the relevant tissue, and by the beam width. The total pulse duration and the average laser power determine the total energy delivered to heat up the tissue, and the duty cycle of the pulse train gives the associated spike, or peak, power associated with the average laser power. Preferably, the pulsed energy source energy parameters are selected so that approximately 20 to 40 joules of energy is absorbed by each cubic centimeter of the target tissue. The absorption length is very small in the thin melanin layer in the retinal pigmented epithelium.

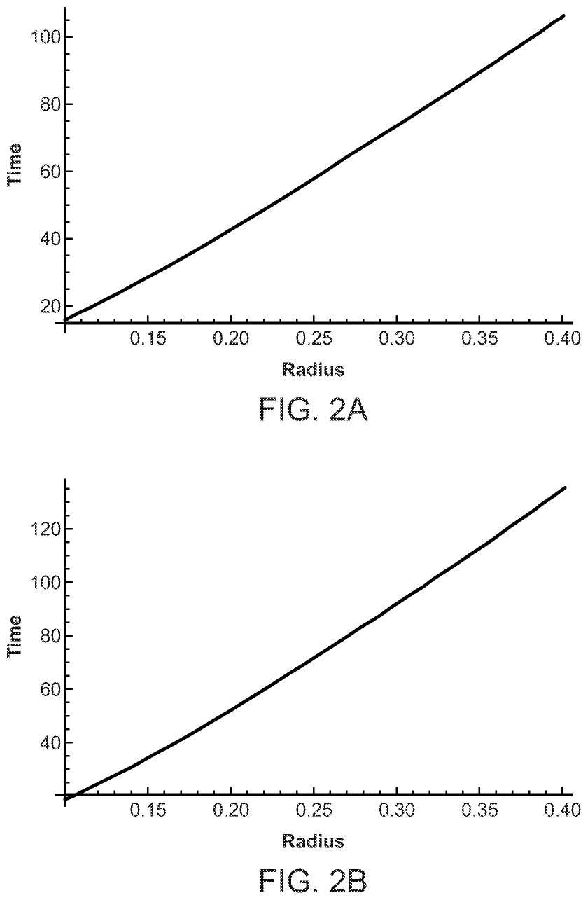

It has been determined that the target tissue can be heated to up to approximately 11° C. for a short period of time, such as less than one second, to create the therapeutic effect of the invention while maintaining the target tissue average temperature to a lower temperature range, such as 1° C. or less over a prolonged period of time, such as several minutes. The selection of the duty cycle and the total pulse train duration provide time intervals in which the heat can dissipate. A duty cycle of less than 10%, and preferably between 2.5% and 5%, with a total pulse duration of between 100 milliseconds and 600 milliseconds has been found to be effective. FIGS. 2A and 2B illustrate the time to decay from 10° C. to 1° C. for a laser source having a radius of between 0.1 cm and 0.4 cm with the wavelength being 880 nm in FIG. 2A and 1000 nm in FIG. 2B. It can be seen that the time to decay is less when using a wavelength of 880 nm, but either wavelength falls within the acceptable requirements and operating parameters to achieve the benefits of the present invention while not causing permanent tissue damage.

It has been found that the average temperature rise of the desired target region increasing at least 6° C. and up to 11° C., and preferably approximately 10° C., during the total irradiation period results in HSP activation. The control of the target tissue temperature is determined by choosing source and target parameters such that the Arrhenius integral for HSP activation is larger than 1, while at the same time assuring compliance with the conservative FDA/FCC requirements for avoiding damage or a damage Arrhenius integral being less than 1.

In order to meet the conservative FDA/FCC constraints to avoid permanent tissue damage, for light beams and other electromagnetic radiation sources, the average temperature rise of the target tissue over any six-minute period is 1° C. or less. FIGS. 2A and 2B above illustrate the typical decay times required for the temperature in the heated target region to decrease by thermal diffusion from a temperature rise of approximately 10° C. to 1° C. as can be seen in FIG. 2A when the wavelength is 880 nm and the source diameter is 1 millimeter, the temperature decay time is 16 seconds. The temperature decay time is 107 seconds when the source diameter is 4 mm. As shown in FIG. 2B, when the wavelength is 1000 nm, the temperature decay time is 18 seconds when the source diameter is 1 mm and 136 seconds when the source diameter is 4 mm. This is well within the time of the average temperature rise being maintained over the course of several minutes, such as 6 minutes or less. While the target tissue's temperature is raised, such as to approximately 10° C., very quickly, such as in a fraction of a second during the application of the energy source to the tissue, the relatively low duty cycle provides relatively long periods of time between the pulses of energy applied to the tissue and the relatively short pulse train duration ensure sufficient temperature diffusion and decay within a relatively short period of time comprising several minutes, such as 6 minutes or less, that there is no permanent tissue damage.

The pulse train mode of energy delivery has a distinct advantage over a single pulse or gradual mode of energy delivery, as far as the activation of remedial HSPs and the facilitation of protein repair is concerned. There are two considerations that enter into this advantage. First, a big advantage for HSP activation and protein repair in an SDM energy delivery mode comes from producing a spike temperature of the order of 10° C. This large rise in temperature has a big impact on the Arrhenius integrals that describe quantitatively the number of HSPs that are activated and the rate of water diffusion into the proteins that facilitates protein repair. This is because the temperature enters into an exponential that has a big amplification effect. Second, it is important that the temperature rise not remain at the high value (10° C. or more) for long, because then it would violate the FDA and FCC requirements that over periods of minutes the average temperature rise must be less than 1° C.

An SDM mode of energy delivery uniquely satisfies both of these foregoing considerations by judicious choice of the power, pulse time, pulse interval, and the volume of the target region to be treated. The volume of the treatment region enters because the temperature must decay from its high value of the order of 10° C. fairly rapidly in order for the long term average temperature rise not to exceed the long term FDA/FCC limit of 1° C. or less for electromagnetic radiation energy sources.

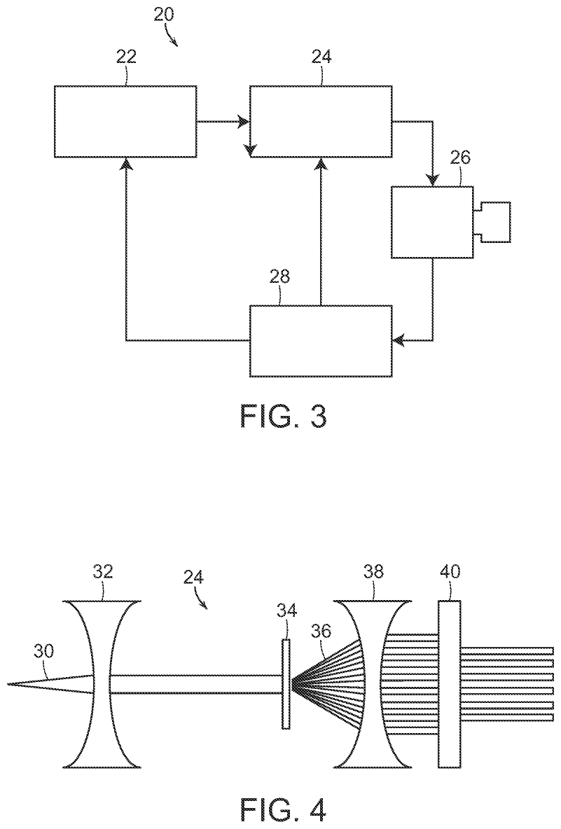

With reference now to FIG. 3, a schematic diagram is shown of a system for generating electromagnetic energy radiation, such as laser light, including SDM. The system, generally referred to by the reference number 20, includes a laser console 22, such as for example the 810 nm near infrared micropulsed diode laser in the preferred embodiment. The laser generates a laser light beam which is passed through optics, such as an optical lens or mask, or a plurality of optical lenses and/or masks 24 as needed. The laser projector optics 24 pass the shaped light beam to a delivery device 26, for projecting the laser beam light onto the target tissue of the patient. It will be understood that the box labeled 26 can represent both the laser beam projector or delivery device as well as a viewing system/camera, or comprise two different components in use. The viewing system/camera 26 provides feedback to a display monitor 28, which may also include the necessary computerized hardware, data input and controls, etc. for manipulating the laser 22, the optics 24, and/or the projection/viewing components 26.

In one embodiment, a plurality of light beams are generated, each of which has parameters selected so that a target tissue temperature may be controllably raised to therapeutically treat the target tissue without destroying or permanently damaging the target tissue. This may be done, for example, by passing the laser light beam 30 through optics which diffract or otherwise generate a plurality of laser light beams from the single laser light beam 30 having the selected parameters. For example, as illustrated in FIG. 4, the laser light beam 30 may be passed through a collimator lens 32 and then through a mask 34. In a particularly preferred embodiment, the mask 34 comprises a diffraction grating. The mask/diffraction grating 34 produces a geometric object, or more typically a geometric pattern of simultaneously produced multiple laser beams or other geometric objects. This is represented by the multiple laser light beams labeled with reference number 36. Alternatively, the multiple laser beams may be generated by a plurality of fiber optic waveguides. Either method of generating laser beams allows for the creation of a very large number of laser spots simultaneously over a very wide treatment field. In fact, a very high number of laser spots, perhaps numbering in the hundreds even thousands or more could be simultaneously generated to cover a given area of the target tissue, or possibly even the entirety of the target tissue. A wide array of simultaneously applied small separated laser spot applications may be desirable as such avoids certain disadvantages and treatment risks known to be associated with large laser spot applications.

Using optical features with a feature size on par with the wavelength of the laser employed, for example using a diffraction grating, it is possible to take advantage of quantum mechanical effects which permits simultaneous application of a very large number of laser spots for a very large target area. The individual beams produced by such diffraction gratings are all of a similar optical geometry to the input beam, with minimal power variation for each beam. The result is a plurality of laser spots with adequate irradiance to produce harmless yet effective treatment application, simultaneously over a large target area. The present invention also contemplates the use of other geometric objects and patterns generated by other diffractive optical elements.

The laser light passing through the mask 34 diffracts, producing a periodic pattern a distance away from the mask 34, shown by the laser beams labeled 36 in FIG. 4. The single laser beam 30 has thus been formed into hundreds or even thousands of individual laser beams 36 so as to create the desired pattern of spots or other geometric objects. These laser beams 36 may be passed through additional lenses, collimators, etc. 38 and 40 in order to convey the laser beams and form the desired pattern. Such additional lenses, collimators, etc. 38 and 40 can further transform and redirect the laser beams 36 as needed.

Arbitrary patterns can be constructed by controlling the shape, spacing and pattern of the optical mask 34. The pattern and exposure spots can be created and modified arbitrarily as desired according to application requirements by experts in the field of optical engineering. Photolithographic techniques, especially those developed in the field of semiconductor manufacturing, can be used to create the simultaneous geometric pattern of spots or other objects.

The present invention can use a multitude of simultaneously generated therapeutic light beams or spots, such as numbering in the dozens or even hundreds, as the parameters and methodology of the present invention create therapeutically effective yet non-destructive and non-permanently damaging treatment. Although hundreds or even thousands of simultaneous laser beams could be generated and created and formed into patterns to be simultaneously applied to the tissue, due to the requirements of not overheating the tissue, there are constraints on the number of treatment beams which can be simultaneously used in accordance with the present invention. Each individual laser beam or spot requires a minimum average power over a train duration to be effective. However, at the same time, tissue cannot exceed certain temperature rises without becoming damaged. For example, using an 810 nm wavelength laser, the number of simultaneous beams generated and used could number from as few as 1 and up to approximately 100 when a 0.04 (4%) duty cycle and a total train duration of 0.3 seconds (300 milliseconds) is used. The water absorption increases as the wavelength is increased. For shorter wavelengths, e.g., 577 nm, the laser power can be lower. For example, at 577 nm, the power can be lowered by a factor of 4 for the invention to be effective. Accordingly, there can be as few as a single laser spot or up to approximately 400 laser spots when using the 577 nm wavelength laser light, while still not harming or damaging the tissue.

Typically, the system of the present invention incorporates a guidance system to ensure complete and total retinal treatment with retinal photostimulation. Fixation/tracking/registration systems consisting of a fixation target, tracking mechanism, and linked to system operation can be incorporated into the present invention. In a particularly preferred embodiment, the geometric pattern of simultaneous laser spots is sequentially offset so as to achieve confluent and complete treatment of the surface.

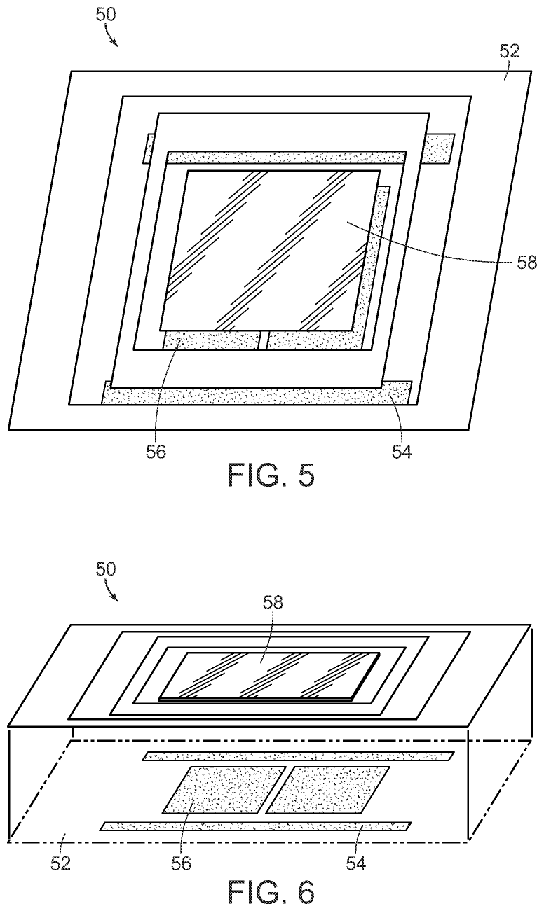

This can be done in a controlled manner using an optical scanning mechanism 50. FIGS. 5 and 6 illustrate an optical scanning mechanism 50 in the form of a MEMS mirror, having a base 52 with electronically actuated controllers 54 and 56 which serve to tilt and pan the mirror 58 as electricity is applied and removed thereto. Applying electricity to the controller 54 and 56 causes the mirror 58 to move, and thus the simultaneous pattern of laser spots or other geometric objects reflected thereon to move accordingly on the retina of the patient. This can be done, for example, in an automated fashion using electronic software program to adjust the optical scanning mechanism 50 until complete coverage of the retina, or at least the portion of the retina desired to be treated, is exposed to the phototherapy. The optical scanning mechanism may also be a small beam diameter scanning galvo mirror system, or similar system, such as that distributed by Thorlabs. Such a system is capable of scanning the lasers in the desired offsetting pattern.

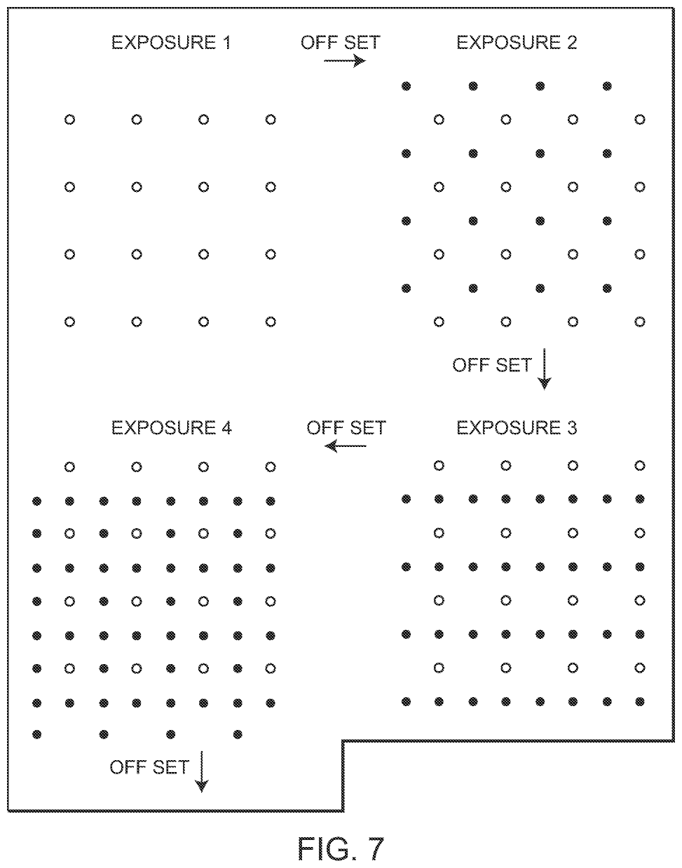

The pattern of spots are offset at each exposure so as to create space between the immediately previous exposure to allow heat dissipation and prevent the possibility of heat damage or tissue destruction. Thus, as illustrated in FIG. 7, the pattern, illustrated for exemplary purposes as a grid of sixteen spots, is offset each exposure such that the laser spots occupy a different space than previous exposures. It will be understood that the diagrammatic use of circles or empty dots as well as filled dots are for diagrammatic purposes only to illustrate previous and subsequent exposures of the pattern of spots to the area, in accordance with the present invention. The spacing of the laser spots prevents overheating and damage to the tissue. It will be understood that this occurs until the entire target tissue to be treated has received phototherapy, or until the desired effect is attained. This can be done, for example, by applying electrostatic torque to a micromachined mirror, as illustrated in FIGS. 5 and 6. By combining the use of small laser spots separated by exposure free areas, prevents heat accumulation, and grids with a large number of spots per side, it is possible to atraumatically and invisibly treat large target areas with short exposure durations far more rapidly than is possible with current technologies.

By rapidly and sequentially repeating redirection or offsetting of the entire simultaneously applied grid array of spots or geometric objects, complete coverage of the target, can be achieved rapidly without thermal tissue injury. This offsetting can be determined algorithmically to ensure the fastest treatment time and least risk of damage due to thermal tissue, depending on laser parameters and desired application.

The following has been modeled using the Fraunhoffer Approximation. With a mask having a nine by nine square lattice, with an aperture radius 9 μm, an aperture spacing of 600 μm, using a 890 nm wavelength laser, with a mask-lens separation of 75 mm, and secondary mask size of 2.5 mm by 2.5 mm, the following parameters will yield a grid having nineteen spots per side separated by 133 μm with a spot size radius of 6 μm. The number of exposures “m” required to treat (cover confluently with small spot applications) given desired area side-length “A”, given output pattern spots per square side “n”, separation between spots “R”, spot radius “r” and desired square side length to treat area “A”, can be given by the following formula:

m=A/nRfloor(R/2r)²

With the foregoing setup, one can calculate the number of operations m needed to treat different field areas of exposure. For example, a 3 mm by 3 mm area, which is useful for treatments, would require 98 offsetting operations, requiring a treatment time of approximately thirty seconds. Another example would be a 3 cm×3 cm area, representing the entire human retinal surface. For such a large treatment area, a much larger secondary mask size of 25 mm by 25 mm could be used, yielding a treatment grid of 190 spots per side separated by 133 μm with a spot size radius of 6 μm. Since the secondary mask size was increased by the same factor as the desired treatment area, the number of offsetting operations of approximately 98, and thus treatment time of approximately thirty seconds, is constant.

Of course, the number and size of spots produced in a simultaneous pattern array can be easily and highly varied such that the number of sequential offsetting operations required to complete treatment can be easily adjusted depending on the therapeutic requirements of the given application.

Furthermore, by virtue of the small apertures employed in the diffraction grating or mask, quantum mechanical behavior may be observed which allows for arbitrary distribution of the laser input energy. This would allow for the generation of any arbitrary geometric shapes or patterns, such as a plurality of spots in grid pattern, lines, or any other desired pattern. Other methods of generating geometric shapes or patterns, such as using multiple fiber optical fibers or microlenses, could also be used in the present invention. Time savings from the use of simultaneous projection of geometric shapes or patterns permits the treatment fields of novel size, such as the 1.2 cm\hat{ }2 area to accomplish whole-retinal treatment, in a single clinical setting or treatment session.

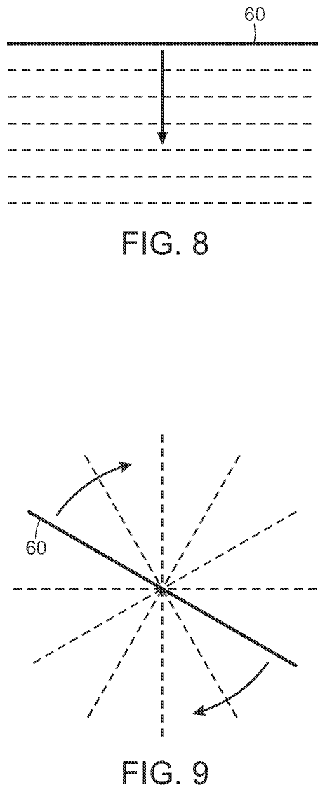

With reference now to FIG. 8, instead of a geometric pattern of small laser spots, the present invention contemplates use of other geometric objects or patterns. For example, a single line 60 of laser light, formed by the continuously or by means of a series of closely spaced spots, can be created. An offsetting optical scanning mechanism can be used to sequentially scan the line over an area, illustrated by the downward arrow in FIG. 8.

With reference now to FIG. 9, the same geometric object of a line 60 can be rotated, as illustrated by the arrows, so as to create a circular field of phototherapy. The potential negative of this approach, however, is that the central area will be repeatedly exposed, and could reach unacceptable temperatures. This could be overcome, however, by increasing the time between exposures, or creating a gap in the line such that the central area is not exposed.

The field of photobiology reveals that different biologic effects may be achieved by exposing target tissues to lasers of different wavelengths. The same may also be achieved by consecutively applying multiple lasers of either different or the same wavelength in sequence with variable time periods of separation and/or with different irradiant energies. The present invention anticipates the use of multiple laser, light or radiant wavelengths (or modes) applied simultaneously or in sequence to maximize or customize the desired treatment effects. This method also minimizes potential detrimental effects. The optical methods and systems illustrated and described above provide simultaneous or sequential application of multiple wavelengths.

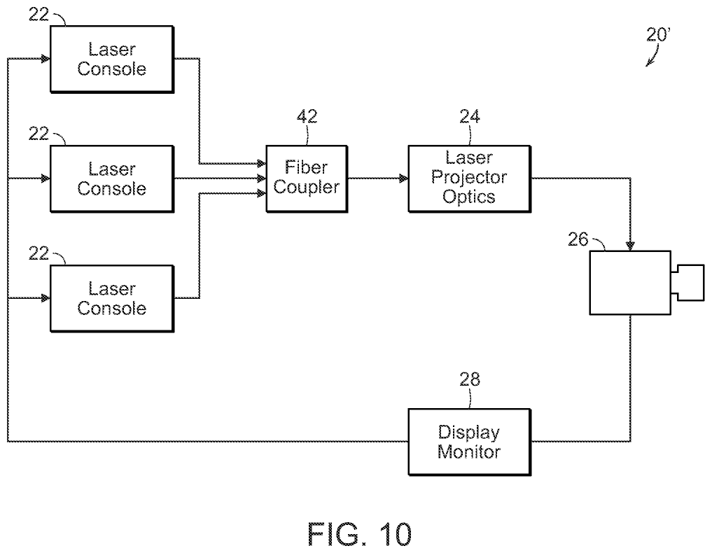

FIG. 10 illustrates diagrammatically a system which couples multiple treatment light sources into the pattern-generating optical subassembly described above. Specifically, this system 20′ is similar to the system 20 described in FIG. 3 above. The primary differences between the alternate system 20′ and the earlier described system 20 is the inclusion of a plurality of laser consoles, the outputs of which are each fed into a fiber coupler 42. Each laser console may supply a laser light beam having different parameters, such as of a different wavelength. The fiber coupler produces a single output that is passed into the laser projector optics 24 as described in the earlier system. The coupling of the plurality of laser consoles 22 into a single optical fiber is achieved with a fiber coupler 42 as is known in the art. Other known mechanisms for combining multiple light sources are available and may be used to replace the fiber coupler described herein.

In this system 20′ the multiple light sources 22 follow a similar path as described in the earlier system 20, i.e., collimated, diffracted, recollimated, and directed to the projector device and/or tissue. In this alternate system 20′ the diffractive element must function differently than described earlier depending upon the wavelength of light passing through, which results in a slightly varying pattern. The variation is linear with the wavelength of the light source being diffracted. In general, the difference in the diffraction angles is small enough that the different, overlapping patterns may be directed along the same optical path through the projector device 26 to the tissue for treatment.

Since the resulting pattern will vary slightly for each wavelength, a sequential offsetting to achieve complete coverage will be different for each wavelength. This sequential offsetting can be accomplished in two modes. In the first mode, all wavelengths of light are applied simultaneously without identical coverage. An offsetting steering pattern to achieve complete coverage for one of the multiple wavelengths is used. Thus, while the light of the selected wavelength achieves complete coverage of the tissue, the application of the other wavelengths achieves either incomplete or overlapping coverage of the tissue. The second mode sequentially applies each light source of a varying wavelength with the proper steering pattern to achieve complete coverage of the tissue for that particular wavelength. This mode excludes the possibility of simultaneous treatment using multiple wavelengths, but allows the optical method to achieve identical coverage for each wavelength. This avoids either incomplete or overlapping coverage for any of the optical wavelengths.

These modes may also be mixed and matched. For example, two wavelengths may be applied simultaneously with one wavelength achieving complete coverage and the other achieving incomplete or overlapping coverage, followed by a third wavelength applied sequentially and achieving complete coverage.

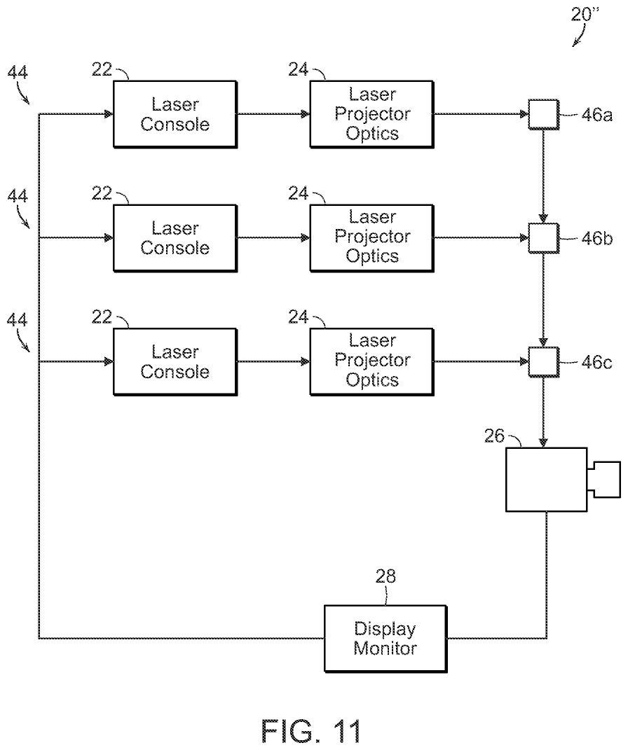

FIG. 11 illustrates diagrammatically yet another alternate embodiment of the inventive system 20″. This system 20″ is configured generally the same as the system 20 depicted in FIG. 3. The main difference resides in the inclusion of multiple pattern-generating subassembly channels tuned to a specific wavelength of the light source. Multiple laser consoles 22 are arranged in parallel with each one leading directly into its own laser projector optics 24. The laser projector optics of each channel 44a, 44b, 44c comprise a collimator 32, mask or diffraction grating 34 and recollimators 38, 40 as described in connection with FIG. 4 above—the entire set of optics tuned for the specific wavelength generated by the corresponding laser console 22. The output from each set of optics 24 is then directed to a beam splitter 46 for combination with the other wavelengths. It is known by those skilled in the art that a beam splitter used in reverse can be used to combine multiple beams of light into a single output. The combined channel output from the final beam splitter 46c is then directed through the projector device 26.

In this system 20″ the optical elements for each channel are tuned to produce the exact specified pattern for that channel's wavelength. Consequently, when all channels are combined and properly aligned a single steering pattern may be used to achieve complete coverage of the tissue for all wavelengths. The system 20″ may use as many channels 44a, 44b, 44c, etc. and beam splitters 46a, 46b, 46c, etc. as there are wavelengths of light being used in the treatment.

Implementation of the system 20″ may take advantage of different symmetries to reduce the number of alignment constraints. For example, the proposed grid patterns are periodic in two dimensions and steered in two dimensions to achieve complete coverage. As a result, if the patterns for each channel are identical as specified, the actual pattern of each channel would not need to be aligned for the same steering pattern to achieve complete coverage for all wavelengths. Each channel would only need to be aligned optically to achieve an efficient combination.

In system 20″, each channel begins with a light source 22, which could be from an optical fiber as in other embodiments of the pattern-generating subassembly. This light source 22 is directed to the optical assembly 24 for collimation, diffraction, recollimation and directed into the beam splitter which combines the channel with the main output.

It will be understood that the laser light generating systems illustrated in FIGS. 3-11 are exemplary. Other devices and systems can be utilized to generate a source of SDM laser light which can be operably passed through to a projector device and steered.

The proposed treatment with a train of electromagnetic pulses has two major advantages over earlier treatments that incorporate a single short or sustained (long) pulse. First, the short (preferably subsecond) individual pulses in the train activate cellular reset mechanisms like HSP activation with larger reaction rate constants than those operating at longer (minute or hour) time scales. Secondly, the repeated pulses in the treatment provide large thermal spikes (on the order of 10,000) that allow the cell's repair system to more rapidly surmount the activation energy barrier that separates a dysfunctional cellular state from the desired functional state. The net result is a “lowered therapeutic threshold” in the sense that a lower applied average power and total applied energy can be used to achieve the desired treatment goal.

Power limitations in current micropulsed diode lasers require fairly long exposure duration. The longer the exposure, the more important the center-spot heat dissipating ability toward the unexposed tissue at the margins of the laser spot. Thus, the micropulsed laser light beam of an 810 nm diode laser should have an exposure envelope duration of 500 milliseconds or less, and preferably approximately 300 milliseconds. Of course, if micropulsed diode lasers become more powerful, the exposure duration should be lessened accordingly.

Aside from power limitations, another parameter of the present invention is the duty cycle, or the frequency of the train of micropulses, or the length of the thermal relaxation time between consecutive pulses. It has been found that the use of a 10% duty cycle or higher adjusted to deliver micropulsed laser at similar irradiance at similar MPE levels significantly increase the risk of lethal cell injury. However, duty cycles of less than 10%, and preferably 2%-5% demonstrate adequate thermal rise and treatment at the level of the MPE cell to stimulate a biological response, but remain below the level expected to produce lethal cell injury. The lower the duty cycle, however, the exposure envelope duration increases, and in some instances can exceed 500 milliseconds.

Each micropulse lasts a fraction of a millisecond, typically between 50 microseconds to 100 microseconds in duration. Thus, for the exposure envelope duration of 300-500 milliseconds, and at a duty cycle of less than 5%, there is a significant amount of wasted time between micropulses to allow the thermal relaxation time between consecutive pulses. Typically, a delay of between 1 and 3 milliseconds, and preferably approximately 2 milliseconds, of thermal relaxation time is needed between consecutive pulses. For adequate treatment, the cells are typically exposed or hit between 50-200 times, and preferably between 75-150 at each location, and with the 1-3 milliseconds of relaxation or interval time, the total time in accordance with the embodiments described above to treat a given area which is being exposed to the laser spots is usually less than one second, such as between 100 milliseconds and 600 milliseconds on average. The thermal relaxation time is required so as not to overheat the cells within that location or spot and so as to prevent the cells from being damaged or destroyed. While time periods of 100-600 milliseconds do not seem long, given the small size of the laser spots and the need to treat a relatively large area of the target tissue, treating the entire target tissue take a significant amount of time, particularly for a patient who is undergoing treatment.

Moreover, the target tissue previously treated with the micropulse of the energy must be allowed to dissipate the heat created by the energy application in order not to exceed a predetermined upper temperature level which could permanently damage or even destroy the cells of the target tissue. Typically, the area or volume of target tissue to be treated is much larger than the area or volume of target tissue which is treated at any given moment by the energy sources, even if multiple beams of energy are created and applied to the target tissue.

Accordingly, the present invention may utilize the interval between consecutive applications to the same location to apply energy to a second treatment area, or additional areas, of the target tissue that is spaced apart from the first treatment area. The pulsed energy is returned to the first treatment location, or previous treatment locations, within the predetermined interval of time so as to provide sufficient thermal relaxation time between consecutive pulses, yet also sufficiently treat the cells in those locations or areas properly by sufficiently increasing the temperature of those cells over time by repeatedly applying the energy to that location in order to achieve the desired therapeutic benefits of the invention.

It is important to return to a previously treated location within a predetermined amount of time to allow the area to cool down sufficiently during that time, but also to treat it within the necessary window of time. In the case of the laser light pulsed energy applications, the laser light is returned to the previously treated location within one to three milliseconds, and preferably approximately two milliseconds, as one cannot wait one or two seconds and then return to a previously treated area that has not yet received the full treatment necessary, as the treatment will not be as effective or perhaps not effective at all. However, during that interval of time, typically approximately 2 milliseconds, at least one other area, and typically multiple areas, can be treated with a laser light application as the laser light pulses are typically 50 seconds to 100 microseconds in duration. This is referred to herein as microshifting. The number of additional areas which can be treated is limited only by the micropulse duration and the ability to controllably move the light beams from one area to another.

Currently, approximately four additional areas which are sufficiently spaced apart from one another can be treated during the thermal relaxation intervals beginning with a first treatment area when using laser light. Thus, multiple areas can be treated, at least partially, during the 200-500 millisecond exposure envelope for the first area. Thus, in a single interval of time, instead of only 100 simultaneous light spots being applied to a treatment area, approximately 500 light spots can be applied during that interval of time in different treatment areas. This would be the case, for example, for a laser light beam having a wavelength of 810 nm. For shorter wavelengths, such as 572 nm, even a greater number of individual locations can be exposed to the laser beams to create light spots. Thus, instead of a maximum of approximately 400 simultaneous spots, approximately 2,000 spots could be covered during the interval between micropulse treatments to a given area or location. Typically each location has between 50-200, and more typically between 75-150, light applications applied thereto over the course of the exposure envelope duration (typically 200-500 milliseconds) to achieve the desired treatment. In accordance with an embodiment of the present invention, the laser light would be reapplied to previously treated areas in sequence during the relaxation time intervals for each area or location. This would occur repeatedly until a predetermined number of laser light applications to each area to be treated have been achieved.

The treatment areas must be separated by at least a predetermined minimum distance to enable thermal relaxation and dissipation and avoid thermal tissue damage. The pulsed energy parameters including wavelength or frequency, duty cycle and pulse train duration are selected so as to raise the target tissue temperature up to 11° C., such as between approximately 6°−11° C., during application of the pulsed energy source to the target tissue to achieve a therapeutic effect, such as by stimulating HSP production within the cells. However, the cells of the target tissue must be given a period of time to dissipate the heat such that the average temperature rise of the tissue over several minutes is maintained at or below a predetermined level, such as 1° C. or less, over several minutes so as not to permanently damage the target tissue.

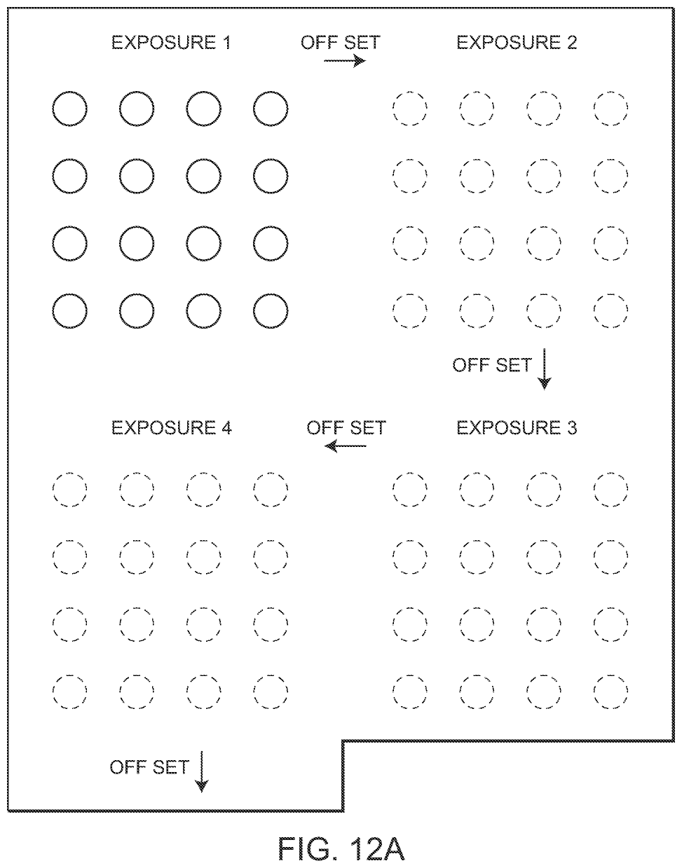

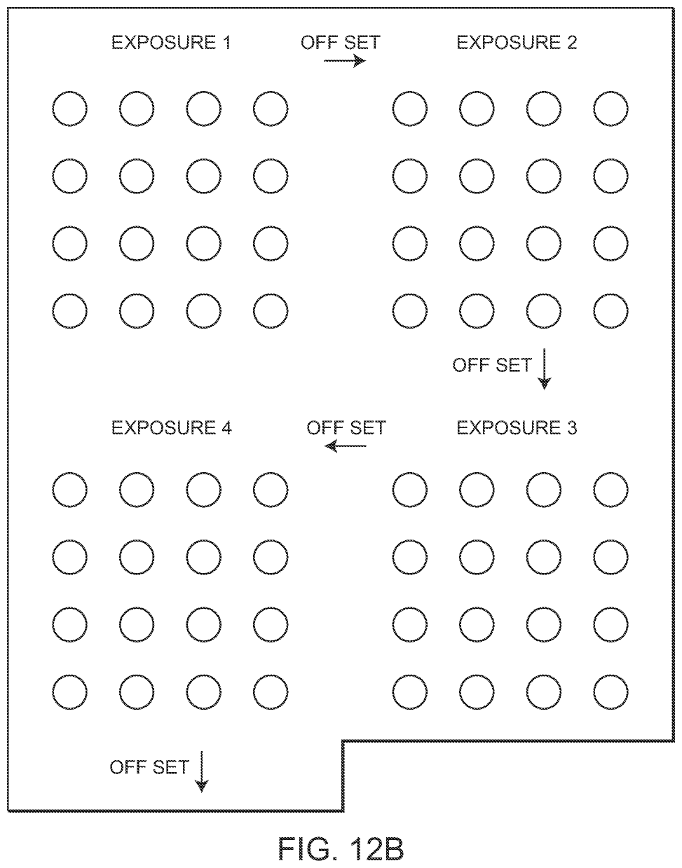

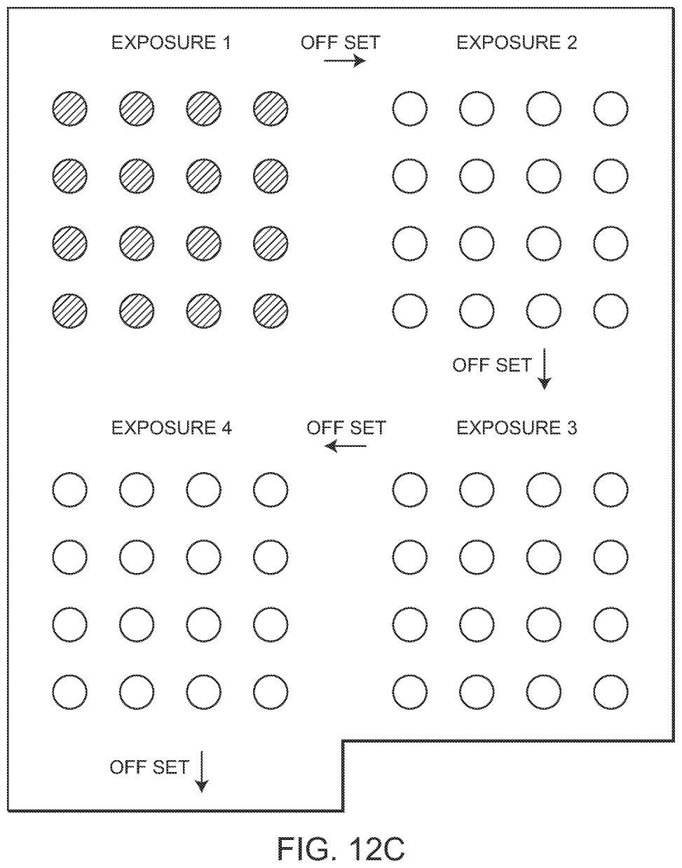

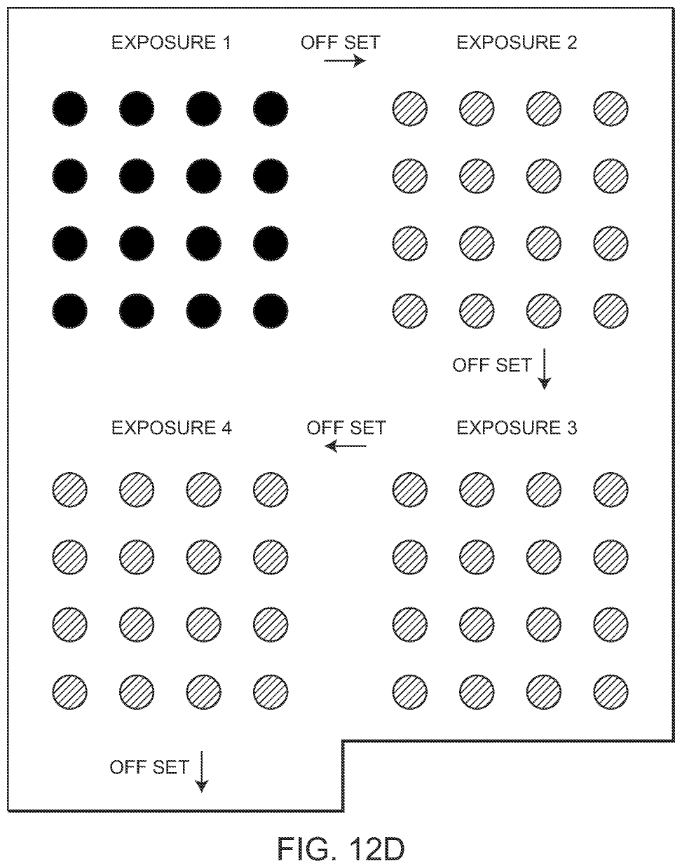

This is diagrammatically illustrated in FIGS. 12A-12D. FIG. 12A illustrates with solid circles a first area having energy beams, such as laser light beams, applied thereto as a first application. The beams are controllably offset or microshifted to a second exposure area, followed by a third exposure area and a fourth exposure area, as illustrated in FIG. 12B, until the locations in the first exposure area need to be re-treated by having beams applied thereto again within the thermal relaxation time interval. The locations within the first exposure area would then have energy beams reapplied thereto, as illustrated in FIG. 12C. Secondary or subsequent exposures would occur in each exposure area, as illustrated in FIG. 12D by the increasingly shaded dots or circles until the desired number of exposures or hits or applications of energy to the target tissue area has been achieved to therapeutically treat these areas, diagrammatically illustrated by the blackened circles in exposure area 1 in FIG. 12D. When a first or previous exposure area has been completed treated, this enables the system to add an additional exposure area, which process is repeated until the entire area to be treated has been fully treated. It should be understood that the use of solid circles, broken line circles, partially shaded circles, and fully shaded circles are for explanatory purposes only, as in fact the exposure of the energy or laser light in accordance with the present invention is invisible and non-detectable to both the human eye as well as known detection devices and techniques.

Adjacent exposure areas must be separated by at least a predetermined minimum distance to avoid thermal tissue damage. Such distance is at least 0.5 diameter away from the immediately preceding treated location or area, and more preferably between 1-2 diameters away. Such spacing relates to the actually treated locations in a previous exposure area. It is contemplated by the present invention that a relatively large area may actually include multiple exposure areas therein which are offset in a different manner than that illustrated in FIG. 12. For example, the exposure areas could comprise the thin lines illustrated in FIGS. 8 and 9, which would be repeatedly exposed in sequence until all of the necessary areas were fully exposed and treated. In accordance with the present invention, the time required to treat that area to be treated is significantly reduced, such as by a factor of 4 or 5 times, such that a single treatment session takes much less time for the medical provider and the patient need not be in discomfort for as long of a period of time.

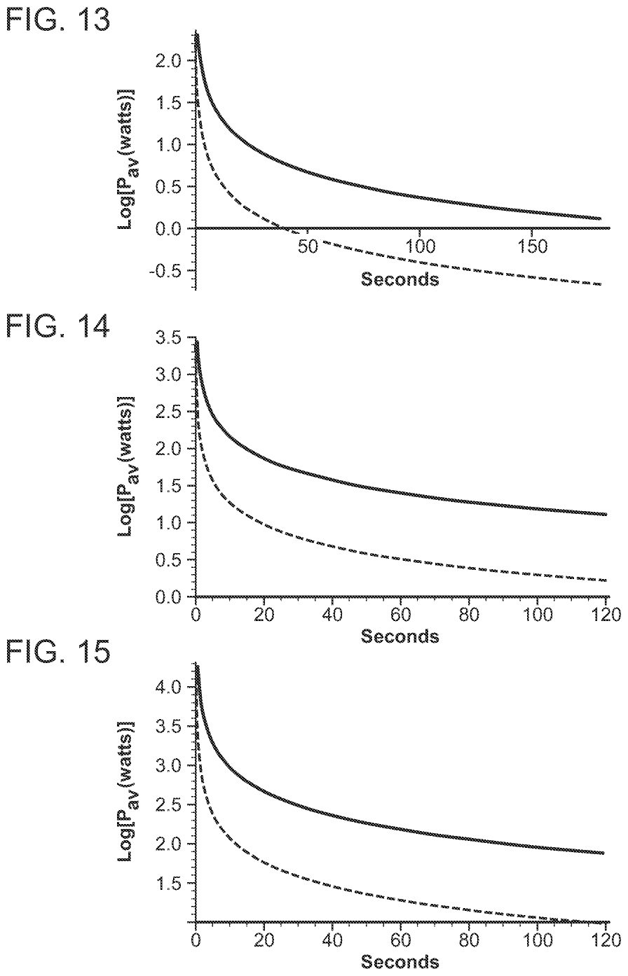

In accordance with this embodiment of the invention of applying one or more treatment beams at once, and moving the treatment beams to a series of new locations, then bringing the beams back to re-treat the same location or area repeatedly has been found to also require less power compared to the methodology of keeping the beams in the same locations or area during the entire exposure envelope duration. With reference to FIGS. 13-15, there is a linear relationship between the pulse length and the power necessary, but there is a logarithmic relationship between the heat generated.

With reference to FIG. 13, a graph is provided wherein the x-axis represents the Log of the average power in watts of a laser and the y-axis represents the treatment time, in seconds. The lower curve is for panmacular treatment and the upper curve is for panretinal treatment. This would be for a laser light beam having a micropulse time of 50 microseconds, a period of 2 milliseconds of time between pulses, and duration of train on a spot of 300 milliseconds. The areas of each retinal spot are 100 microns, and the laser power for these 100 micron retinal spots is 0.74 watts. The panmacular area is 0.55², requiring 7,000 panmacular spots total, and the panretinal area is 3.30², requiring 42,000 laser spots for full coverage. Each RPE spot requires a minimum energy in order for its reset mechanism to be adequately activated, in accordance with the present invention, namely, 38.85 joules for panmacular and 233.1 joules for panretinal. As would be expected, the shorter the treatment time, the larger the required average power. However, there is an upper limit on the allowable average power, which limits how short the treatment time can be.

As mentioned above, there are not only power constraints with respect to the laser light available and used, but also the amount of power that can be applied to the eye without damaging eye tissue. For example, temperature rise in the lens of the eye is limited, such as between 4° C. so as not to overheat and damage the lens, such as causing cataracts. Thus, an average power of 7.52 watts could elevate the lens temperature to approximately 4° C. This limitation in power increases the minimum treatment time.

However, with reference to FIG. 14, the total power per pulse required is less in the microshift case of repeatedly and sequentially moving the laser spots and returning to prior treated locations, so that the total energy delivered and the total average power during the treatment time is the same. FIGS. 14 and 15 show how the total power depends on treatment time. This is displayed in FIG. 14 for panmacular treatment, and in FIG. 15 for panretinal treatment. The upper, solid line or curve represents the embodiment where there are no microshifts taking advantage of the thermal relaxation time interval, such as described and illustrated in FIG. 7, whereas the lower dashed line represents the situation for such microshifts, as described and illustrated in FIG. 12. FIGS. 14 and 15 show that for a given treatment time, the peak total power is less with microshifts than without microshifts. This means that less power is required for a given treatment time using the microshifting embodiment of the present invention. Alternatively, the allowable peak power can be advantageously used, reducing the overall treatment time.

Thus, in accordance with FIGS. 13-15, a log power of 1.0 (10 watts) would require a total treatment time of 20 seconds using the microshifting embodiment of the present invention, as described herein. It would take more than 2 minutes of time without the microshifts, and instead leaving the micropulsed light beams in the same location or area during the entire treatment envelope duration. There is a minimum treatment time according to the wattage. However, this treatment time with microshifting is much less than without microshifting. As the laser power required is much less with the microshifting, it is possible to increase the power in some instances in order to reduce the treatment time for a given desired retinal treatment area. The product of the treatment time and the average power is fixed for a given treatment area in order to achieve the therapeutic treatment in accordance with the present invention. This could be implemented, for example, by applying a higher number of therapeutic laser light beams or spots simultaneously at a reduced power. Of course, since the parameters of the laser light are selected to be therapeutically effective yet not destructive or permanently damaging to the cells, no guidance or tracking beams are required, only the treatment beams as all areas can be treated in accordance with the present invention.

In accordance with the microshifting technique described above, the shifting or steering of the pattern of light beams may be done by use of an optical scanning mechanism, such as that illustrated and described in connection with FIGS. 5 and 6.

Although the present invention is described for use in connection with a micropulsed laser, theoretically a continuous wave laser could potentially be used instead of a micropulsed laser. However, with the continuous wave laser, there is concern of overheating as the laser is moved from location to location in that the laser does not stop and there could be heat leakage and overheating between treatment areas. Thus, while it is theoretically possible to use a continuous wave laser, in practice it is not ideal and the micropulsed laser is preferred.

As mentioned above, the controlled manner of applying energy to the target tissue is intended to raise the temperature of the target tissue to therapeutically treat the target tissue without destroying or permanently damaging the target tissue. It is believed that such heating activates HSPs and that the thermally activated HSPs work to reset the diseased tissue to a healthy condition, such as by removing and/or repairing damaged proteins. It is believed by the inventors that maximizing such HSP activation improves the therapeutic effect on the targeted tissue. As such, understanding the behavior and activation of HSPs and HSP system species, their generation and activation, temperature ranges for activating HSPs and time frames of the HSP activation or generation and deactivation can be utilized to optimize the heat treatment of the biological target tissue.

As mentioned above, the target tissue is heated by the pulsed energy for a short period of time, such as ten seconds or less, and typically less than one second, such as between 100 milliseconds and 600 milliseconds. The time that the energy is actually applied to the target tissue is typically much less than this in order to provide intervals of time for heat relaxation so that the target tissue does not overheat and become damaged or destroyed. For example, as mentioned above, laser light pulses may last on the order of microseconds with several milliseconds of intervals of relaxed time between laser light pulses.

Thus, understanding the sub-second behaviors of HSPs can be important to the present invention. The thermal activation of the HSPs in SDM is typically described by an associated Arrhenius integral,

Ω=∫dt A exp[−E/k_{B}T(t)] [1]

where the integral is over the treatment time and

A is the Arrhenius rate constant for HSP activation

E is the activation energy

T(t) is the temperature of the thin RPE layer, including the laser-induced temperature rise

The laser-induced temperature rise—and therefore the activation Arrhenius integral—depends on both the treatment parameters (e.g., laser power, duty cycle, total train duration) and on the RPE properties (e.g., absorption coefficients, density of HSPs). It has been found clinically that effective SDM treatment is obtained when the Arrhenius integrals is of the order of unity.

The Arrhenius integral formalism only takes into account a forward reaction, i.e. only the HSP activation reaction, it does not take into account any reverse reactions in which activated HSPs are returned to their inactivated states. For the typical subsecond durations of SDM treatments, this appears to be quite adequate. However, for longer periods of time (e.g. a minute or longer), this formalism is not a good approximation: At these longer times, a whole series of reactions occurs resulting in much smaller effective HSP activation rates. This is the case during the proposed minute or so intervals between SDM applications in the present invention disclosure.

In the published literature, the production and destruction of heat shock proteins (HSPs) in cells over longer durations is usually described by a collection of 9-13 simultaneous mass-balance differential equations that describe the behavior of the various molecular species involved in the life cycle of an HSP molecule. These simultaneous equations are usually solved by computer to show the behavior in time of the HSPs and the other species after the temperature has been suddenly raised.

These equations are all conservation equations based on the reactions of the various molecular species involved in the activity of HSPs To describe the behavior of the HSPs in the minute or so intervals between repeated applications of SDM, we shall use the equations described in M. Rybinski, Z. Szymanska, S. Lasota, A. Gambin (2013) Modeling the efficacy of hyperthermia treatment. Journal of the Royal Society Interface 10, No. 88, 20130527 (Rybinski et al (2013)). The species considered in Rybinski et al (2013) are shown in Table 1.

TABLE 1
HSP system species in Rybinski et al (2013) description:

| | |
|---|---|
| HSP | ubiquitous heat shock protein of molecular weight 70 Da (in |
| | free, activated state) |
| HSF | heat shock (transcription) factor that has no DNA binding |
| | capability |
| HSF₃ | (trimer) heat shock factor capable of binding to DNA, |
| | formed from HSF |
| HSE | heat shock element, a DNA site that initiates transcription |
| | of HSP when bound to HSF₃ |
| mRNA | messenger RNA molecule for producing HSP |
| S | substrate for HSP binding: a damaged protein |
| P | properly folded protein |
| HSP•HSF | a complex of HSP bound to HSF (unactivated HSPs) |
| HSF₃•HSE | a complex of HSF₃ bound to HSE, that induces |
| | transcription and the creation of a new HSP mRNA |
| | molecule |
| HSP•S | a complex of HSP attached to damaged protein (HSP |
| | actively repairing the protein) |

The coupled simultaneous mass conservation equations for these 10 species are summarized below as eqs. [2]-[11]:

d[HSP]/dt=(l₁+k₁₀)[HSPS]+l₂[HSPHSF]+k₄[mRNA]−k₁[S][HSP]−k₂[HSP][HSF]−l₃[HSP][HSF₃]−k₉[HSP] [2]

d{HSF]/dt=l₂[HSPHSF]+2l₃[HSP][HSF₃]+k₆[HSPHSF][S]−k₂[HSP][HSF]−3k₃[HSF]³−l₆[HSPS][HSF] [3]

d[S]/dt=k₁₁{[P]+l₁[HSPS]+l₆[SPS][HSF]−k₁[S][HSP]−k₆[HSPHSF][S] [4]

d[HSPHSF]/dt=k₂[HSP][HSF]+l₆[HSPS][HSF]+l₃[HSP][HSF₃]−l₂[HSPHSF]−k₆[HSPHSF][S] [5]

d[HSPS]/dt=k₁[S][HSP]+k₆[HSPHSF][S]−(l₁+k₁₀[HSPS]−l₆[HSPS][HSF] [6]

d[HSF₃]/dt=k₃[HSF]³+l₇[HSF₃][HSE]−l₃[HSP][HSF₃]−k₇[HSF₃][HSE] [7]

d[HSE]/dt=l₇[HSF₃][HSE]−k₇[HSF₃][HSE] [8]

d[HSF₃HSE]/dt=k₇[HSF₃][HSE]−l₇[HSF₃][HSE] [9]

d[mRNA]/dt=k₈[HSF₃HSE]−k₅[mRNA] [10]

d[P]/dt=k₁₀[HSPS]−k₁₁[P] [11]

In these expressions, [ ] denotes the cellular concentration of the quantity inside the bracket. For Rybinski et al (2013), the initial concentrations at the equilibrium temperature of 310K are given in Table 2.

TABLE 2
Initial values of species at 310 K for a typical cell in arbitrary
units [Rybinski et al (2013)]. The arbitrary units are
chosen by Rybinski et al for computational convenience: to
make the quantities of interest in the range of 0.01-10.

| | |
|---|---|
| [HSP(0)] | 0.308649 |
| [HSF(0)] | 0.150836 |
| [S(0)] | 0.113457 |
| [HSPHSF(0)] | 2.58799 |
| [HSPS(0)] | 1.12631 |
| [HSF₃(0)] | 0.0444747 |
| [HSE(0)] | 0.957419 |
| [HSF₃HSE(0)] | 0.0425809 |
| [mRNA(0)] | 0.114641 |
| [P(0)] | 8.76023 |

The Rybinski et al (2013) rate constants are shown in Table 3.

TABLE 3
Rybinski et al (2013) rate constants giving rates in min⁻¹ for
the arbitrary concentration units of the previous table.

l₁ = 0.0175
k₁ = 1.47
l₂ = 0.0175
k₂ = 1.47
l₃ = 0.020125
k₃ = 0.0805
k₄ = 0.1225
k₅ = 0.0455
k₆ = 0.0805
l₆ = 0.00126
k₇ = 0.1225
l₇ = 0.1225
k₈ = 0.1225
k₉ = 0.0455
k₁₀ = 0.049
k₁₁ = 0.00563271

The initial concentration values of Table 2 and the rate constants of Table 3 were determined by Rybinski et al (2013) to correspond to experimental data on overall HSP system behavior when the temperature was increased on the order of 5° C. for several (e.g. 350) minutes.

Note that the initial concentration of HSPs is 100×0.308649/(8.76023+0.113457+1.12631)}=3.09% of the total number of proteins present in the cell.

Although the rate constants of Table 3 are used by Rybinski et al for T=310+5+31 5K, it is likely that very similar rate constants exist at other temperatures. In this connection, the qualitative behavior of the simulations is similar for a large range of parameters. For convenience, we shall assume that the values of the rate constants in Table 3 are a good approximation for the values at the equilibrium temperature of T=310K.

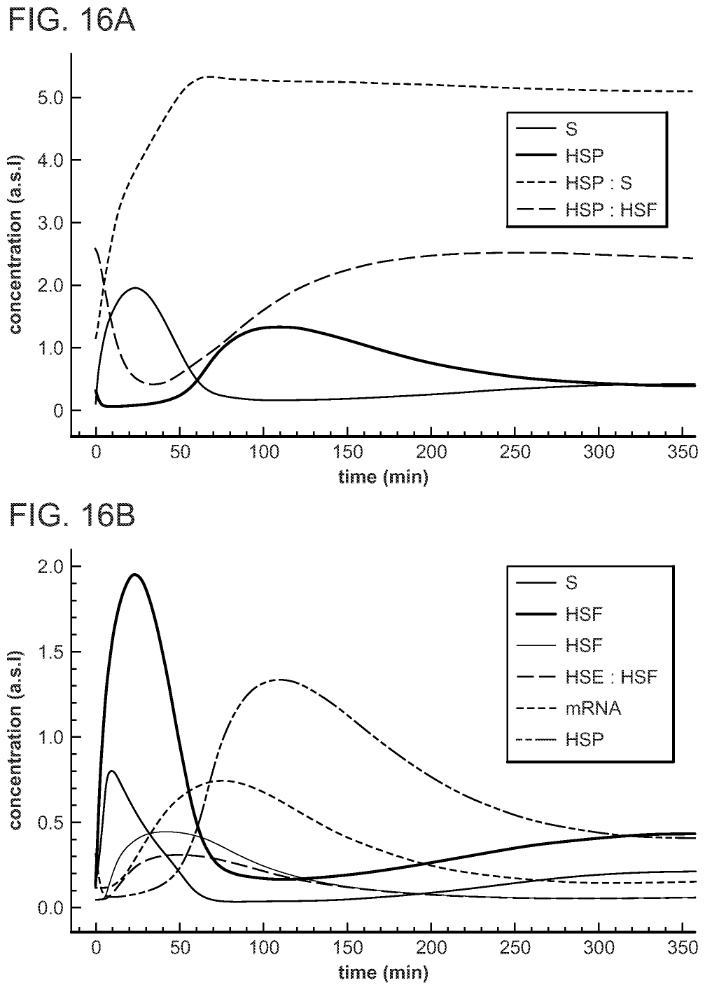

The behavior of the different components in the Rybinski et al cell is displayed in FIG. 16 for 350 minutes for the situation where the temperature is suddenly increased 5K at t=0 from an ambient 310K.

With continuing reference to FIG. 16, the behavior of HSP cellular system components during 350 minutes following a sudden increase in temperature from 37° C. to 42° C. is shown. Here, the concentrations of the components are presented in computationally convenient arbitrary units. S denotes denatured or damaged proteins that are as yet unaffected by HSPs; HSP denotes free (activated) heat shock proteins; HSP:S denotes activated HSPs that are attached to the damaged proteins and performing repair; HSP:HSF denotes (inactive) HSPs that are attached to heat shock factor monomers; HSF denotes a monomer of heat shock factor; HSF₃ denotes a trimer of heat shock factor that can penetrate the nuclear membrane to interact with a heat shock element on the DNA molecule; HSE:HSF₃ denotes a trimer of heat shock factor attached to a heat shock element on the DNA molecule that initiates transcription of a new mRNA molecule; mRNA denotes the messenger RNA molecule that results from the HSE:HSF₃, and that leads to the production of a new (activated) HSP molecule in the cell's cytoplasm.

FIG. 16 shows that initially the concentration of activated HSPs is the result of release of HSPs sequestered in the molecules HSPHSF in the cytoplasm, with the creation of new HSPs from the cell nucleus via mRNA not occurring until 60 minutes after the temperature rise occurs. FIG. 16 also shows that the activated HSPs are very rapidly attached to damaged proteins to begin their repair work. For the cell depicted, the sudden rise in temperature also results in a temporary rise in damaged protein concentration, with the peak in the damaged protein concentration occurring about 30 minutes after the temperature increase.

FIG. 16 shows what the Rybinski et al equations predict for the variation of the 10 different species over a period of 350 minutes. However, the present invention is concerned with SDM application is on the variation of the species over the much shorter O(minute) interval between two applications of SDM at any single retinal locus. It will be understood that the preferred embodiment of SDM in the form of laser light treatment is analyzed and described, but it is applicable to other sources of energy as well.

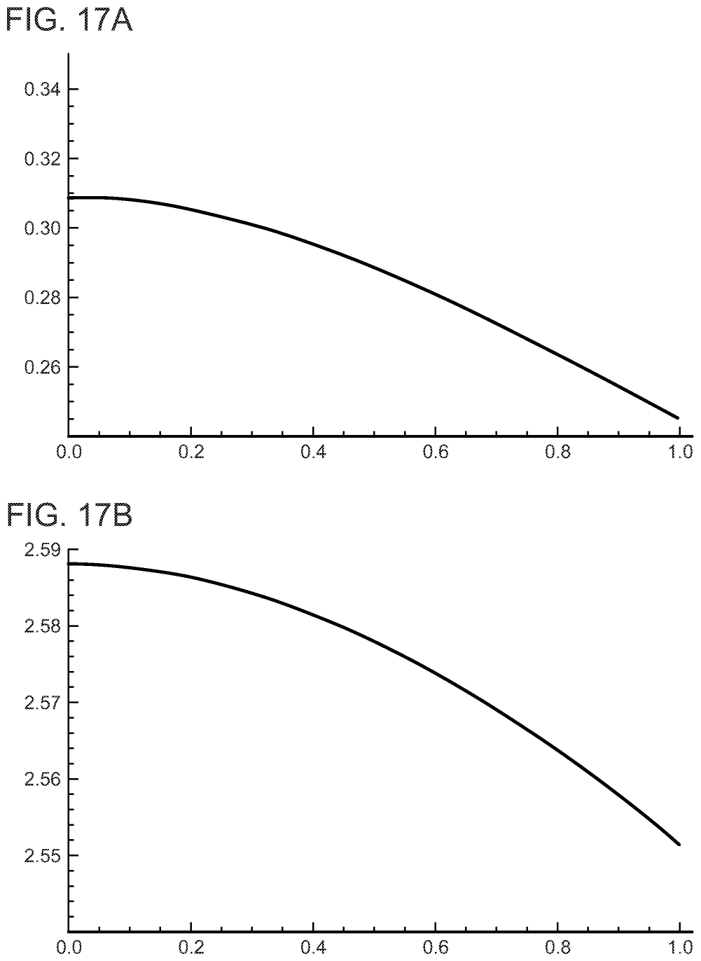

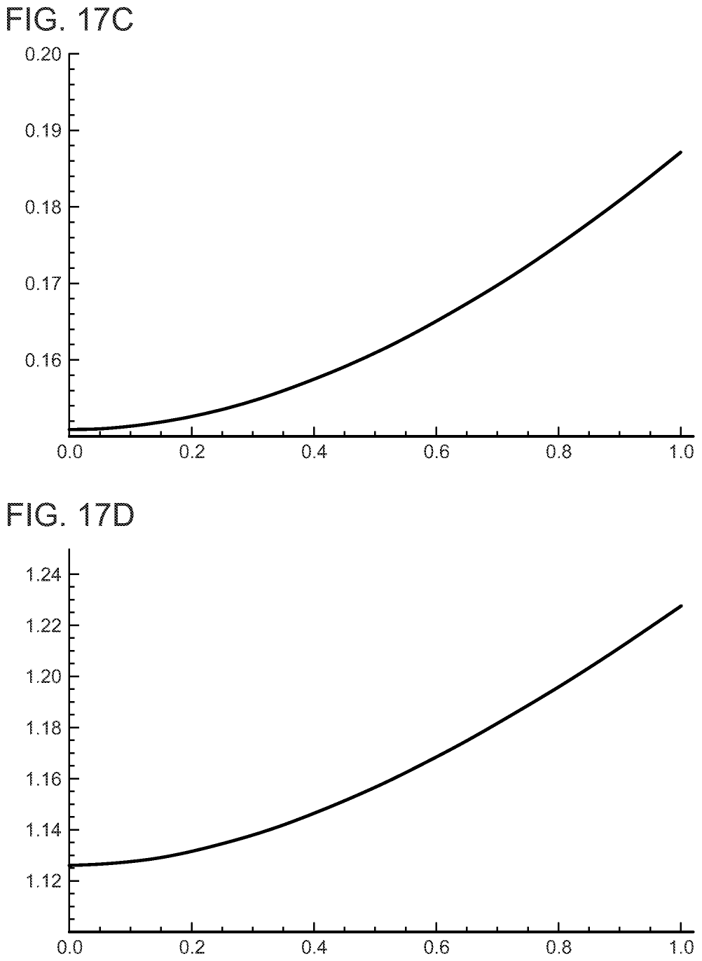

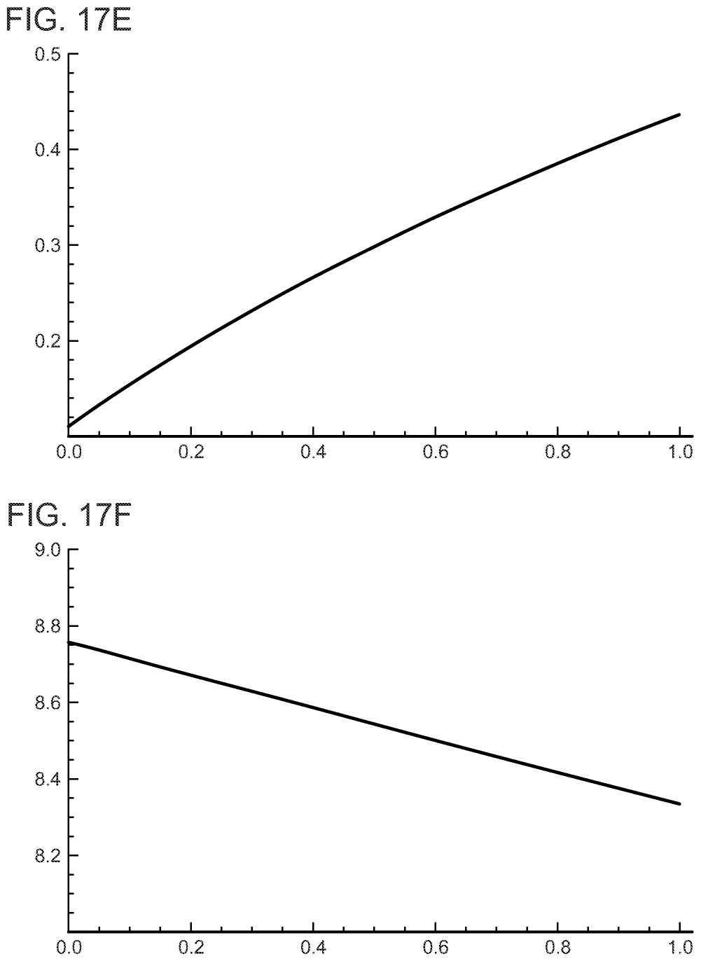

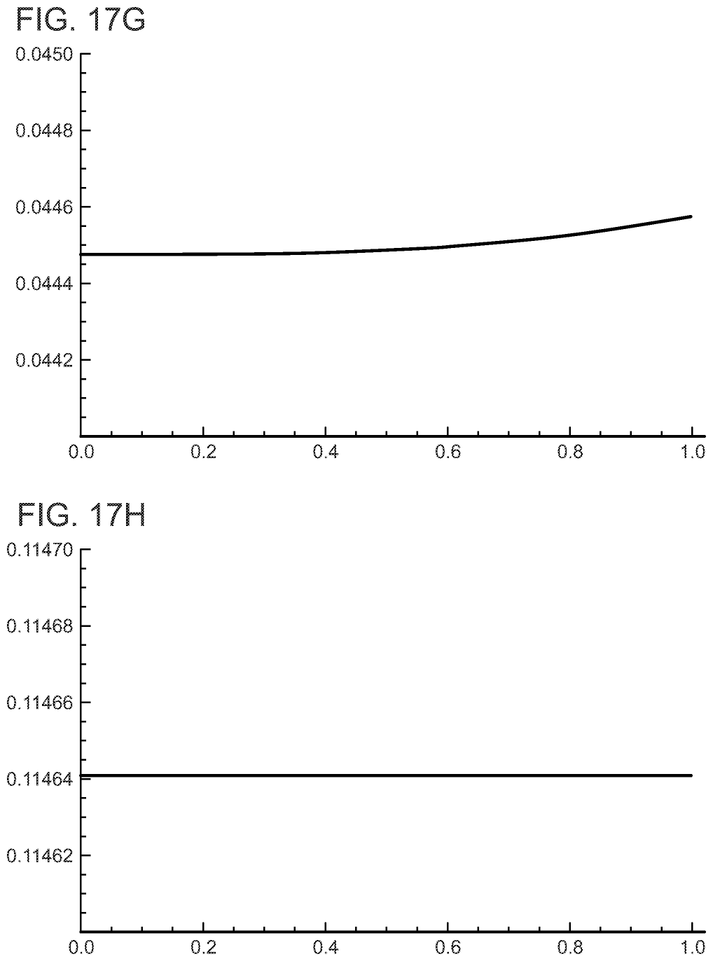

With reference now to FIGS. 17A-17H, the behavior of HSP cellular system components during the first minute following a sudden increase in temperature from 37° C. to 42° C. using the Rybinski et al. (2013) equations with the initial values and rate constants of Tables 2 and 3 are shown. The abscissa denotes time in minutes, and the ordinate shows concentration in the same arbitrary units as in FIG. 17.

FIG. 17 shows that the nuclear source of HSPs plays virtually no role during a 1 minute period, and that the main source of new HSPs in the cytoplasm arises from the release of sequestered HSPs from the reservoir of HSPHSF molecules. It also shows that a good fraction of the newly activated HSPs attach themselves to damaged proteins to begin the repair process.

The initial concentrations in Table 2 are not the equilibrium values of the species, i.e. they do not give d[ . . . ]/dt=0, as evidenced by the curves in FIGS. 16 and 17. The equilibrium values that give d[ . . . ]/dt=0 corresponding to the rate constants of Table 3 are found to be those listed in Table 4.

TABLE 4
Equilibrium values of species in arbitrary units [Rybinski et al
(2013)] corresponding to the rate constants of Table 3. The arbitrary
units are those chosen by Rybinski et al for computational convenience:
to make the quantities of interest in the range of 0.01-10.

| | |
|---|---|
| [HSP(equil)] | 0.315343 |
| [HSF(equil)] | 0.255145 |
| [S(equil)] | 0.542375 |
| [HSPHSF(equil)] | 1.982248 |
| [HSPS(equil)] | 5.05777 |
| [HSF₃(equil)] | 0.210688 |
| [HSE(equil)] | 0.206488 |
| [HSF₃HSE(equil)] | 0.643504 |
| [mRNA(equil)] | 0.1171274 |
| [P(equil)] | 4.39986 |

Note that the equilibrium concentration of HSPs is 100×{0.315343/(4.39986+5.05777+0.542375)}=3.15% of the total number of proteins present in the cell. This is comparable, but less than the anticipated 5%-10% total number of proteins found by other researchers. However, we have not attempted to adjust percentage upwards expecting that the general behavior will not be appreciably changed as indicated by other researchers.

The inventors have found that a first treatment to the target tissue may be performed by repeatedly applying the pulsed energy (e.g., SDM) to the target tissue over a period of time so as to controllably raise a temperature of the target tissue to therapeutically treat the target tissue without destroying or permanently damaging the target tissue. A “treatment” comprises the total number of applications of the pulsed energy to the target tissue over a given period of time, such as dozens or even hundreds of light or other energy applications to the target tissue over a short period of time, such as a period of less than ten seconds, and more typically a period of less than one second, such as 100 milliseconds to 600 milliseconds. This “treatment” controllably raises the temperature of the target tissue to activate the heat shock proteins and related components.

What has been found, however, is that if the application of the pulsed energy to the target tissue is halted for an interval of time, such as an interval of time that exceeds the first period of time comprising the “first treatment”, which may comprise several seconds to several minutes, such as three seconds to three minutes or more preferably ten seconds to ninety seconds, and then a second treatment is performed on the target tissue after the interval of time within a single treatment session or office visit, wherein the second treatment also entails repeatedly reapplying the pulsed energy to the target tissue so as to controllably raise the temperature of the target tissue to therapeutically treat the target tissue without destroying or permanently damaging the target tissue, the amount of activated HSPs and related components in the cells of the target tissue is increased resulting in a more effective overall treatment of the biological tissue. In other words, the first treatment creates a level of heat shock protein activation of the target tissue, and the second treatment increases the level of heat shock protein activation in the target tissue above the level due to the first treatment. Thus, performing multiple treatments to the target tissue of the patient within a single treatment session or office visit enhances the overall treatment of the biological tissue so long as the second or additional treatments are performed after an interval of time which does not exceed several minutes but which is of sufficient length so as to allow temperature relaxation so as not to damage or destroy the target tissue.

This technique may be referred to herein as “stair-stepping” in that the levels of activated HSP production increase with the subsequent treatment or treatments within the same office visit treatment session. This “stair-stepping” technique may be described by a combination of the Arrhenius integral approach for subsecond phenomena with the Rybinski et al. (2013) treatment of intervals between repeated subsecond applications of the SDM or other pulsed energy.

For the proposed stair-stepping SDM (repetitive SDM applications) proposed in this invention disclosure, there are some important differences from the situation depicted in FIG. 16:
SDM can be applied prophylactically to a healthy cell, but oftentimes SDM will be applied to a diseased cell. In that case, the initial concentration of damaged proteins [S(0)] can be larger than given in Table 4. We shall not attempt to account for this, assuming that the qualitative behavior will not be changed.
The duration of a single SDM application is only subseconds, rather than the minutes shown in FIG. 16. The Rybinski et al rate constants are much smaller than the Arrhenius constants: the latter give Arrhenius integrals of the order of unity for subsecond durations, whereas the Rybinski et al rate constants are too small to do that. This is an example of the different effective rate constants that exist when the time scales of interest are different: The Rybinski et al rate constants apply to phenomena occurring over minutes, whereas the Arrhenius rate constants apply to subsecond phenomena.

Accordingly, to analyze what happens in the proposed stair-stepping SDM technique for improving the efficacy of SDM, we shall combine the Arrhenius integral treatment appropriate for the subsecond phenomena with the Rybinski et al (2013) treatment appropriate for the phenomena occurring over the order of a minute interval between repeated SDM applications:
SDM subsecond application described by Arrhenius integral formalism
Interval of O(minute) between SDM applications described by Rybinski et al (2013) equations

Specifically, we consider two successive applications of SDM, each SDM micropulse train having a subsecond duration.
For the short subsecond time scale, we assume that the unactivated HSP's that are the source of the activated (free) HSP's are all contained in the HSPHSF molecules in the cytoplasm. Accordingly, the first SDM application is taken to reduce the cytoplasmic reservoir of unactivated HSPs in the initial HSPHSF molecule population from
   [HSPHSF(equil)] to [HSPHSF(equil)]exp[−Ω],
and to increase the initial HSP molecular population from
   [HSP(equil)] to [HSP(equil)]+[HSPHSF(equil)](1−exp[−Ω])
as well as to increase the initial HSF molecular population from
   [HSF(equil)] to [HSF(equil)]+[HSPHSF(equil)](1−exp[−Ω])
The equilibrium concentrations of all of the other species will be assumed to remain the same after the first SDM application
The Rybinski et al equations are then used to calculate what happens to [HSP] and [HSPHSF] in the interval [symbol] t=O(minute) between the first SDM application and the second SDM application, with the initial values of HSP, HSF and HSPHSF after the first SDM application taken to be
   [HSP(SDM1)]=[HSP(equil)]+[HSPHSF(equil)](1−exp[−Ω])
   [HSF(SDM1)]=[HSF(equil)]+[HSPHSF(equil)](1−exp[−Ω])
and
   [HSPHSF(SDM1)]=[HSPHSF(equil)]exp[−Ω]
For the second application of SDM after the interval [symbol] t, the values of [HSP], [HSF] and {HSPHSF] after the SDM will be taken to be
   [HSP(SDM2)]=[HSP( [symbol] t)]+[HSPHSF( [symbol] t)](1−exp[−Ω])
   [HSF(SDM2)]=[HSF( [symbol] t)]+[HSPHSF( [symbol] t)](1−exp[−Ω])
and
   [HSPHSF(SDM2)]=[HSPHSF( [symbol] t)]exp[−Ω]
where [HSP( [symbol] t)], [HSF( [symbol] t)], and [HSPHSF( [symbol] t)] are the values determined from the Rybinski et al (2013) equations at the time [symbol] t.
Our present interest is in comparing [HSP[SDM2)] with [HSP[SDM1)], to see if the repeated application of SDM at an interval [symbol] t following the first application of SDM has resulted in more activated (free) HSP's in the cytoplasm. The ratio β( [symbol] t, Ω)=[HSP(SDM2)]/[HSP(SDM1)]={[{[HSP( [symbol] t)]+[HSPHSF( [symbol] t)](1−exp[−Ω])}/{[HSP(0)]+[HSPHSF(0)](1−exp[−Ω])}
provides a direct measure of the improvement in the degree of HSP activation for a repeated application of SDM after an interval [symbol] t from the first SDM application.

The HSP and HSPHSF concentrations can vary quite a bit in the interval [symbol] t between SDM applications.

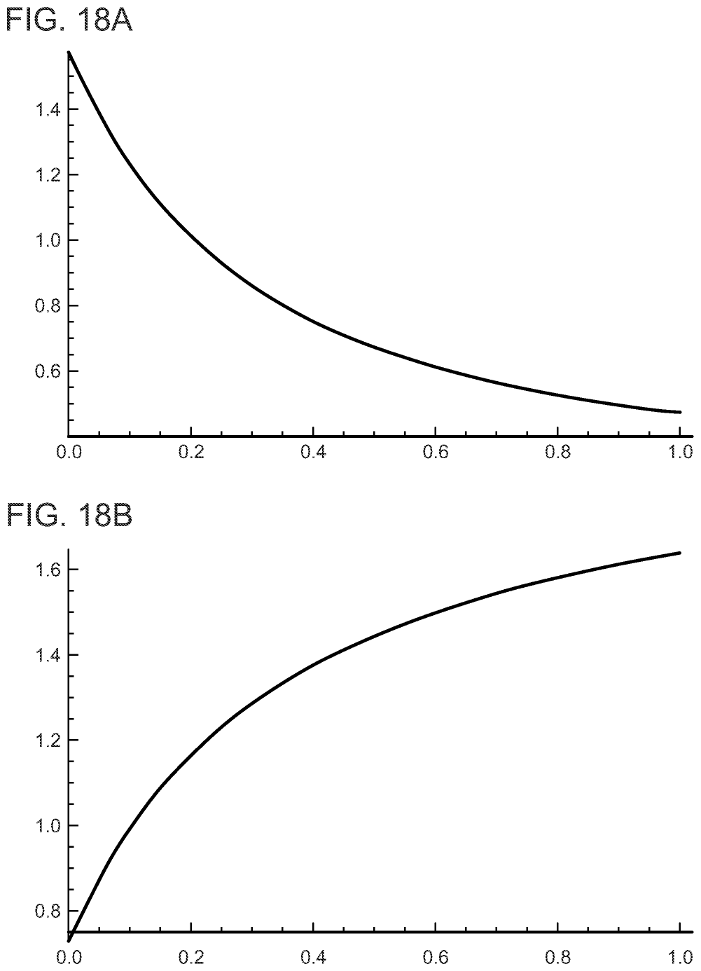

FIGS. 18A and 18B illustrate the variation in the activated concentrations [HSP] and the unactivated HSP in the cytoplasmic reservoir [HSPHSF] during an interval [symbol] t=1 minute between SDM applications when the SDM Arrhenius integral Ω=1 and the equilibrium concentrations are as given in Table 4.

Although only a single repetition (one-step) is treated here, it is apparent that the procedure could be repeated to provide a multiple stair-stepping events as a means of improving the efficacy of SDM, or other therapeutic method involving activation of tissue HSPs.

Effects of varying the magnitude of the Arrhenius integral Ω and interval [symbol] t between two distinct treatments separated by an interval of time are shown by the following examples and results.

Nine examples generated with the procedure described above are presented in the following. All of the examples are of a treatment consisting of two SDM treatments, with the second occurring at a time [symbol] t following the first, and they explore:
The effect of different magnitude Arrhenius integrals Ω in the SDM treatments [Three different Ω's are considered: Ω=0.2, 0.5 and 1.0]
The impact of varying the interval [symbol] t between the two SDM treatments [Three different [symbol] t's are considered: [symbol] t=15 sec., 30 sec., and 60 sec.

As indicated above, the activation Arrhenius integral Ω depends on both the treatment parameters (e.g., laser power, duty cycle, total train duration) and on the RPE properties (e.g., absorption coefficients, density of HSPs).

Table 5 below shows the effect of different Ω (Ω=0.2, 0.5, 1) on the HSP content of a cell when the interval between the two SDM treatments is [symbol] t=1 minute. Here the cell is taken to have the Rybinski et al (2013) equilibrium concentrations for the ten species involved, given in Table 4.

Table 5 shows four HSP concentrations (in the Rybinski et al arbitrary units) each corresponding to four different times:
Before the first SDM treatment: [HSP(equil)]
Immediately after the first SDM application: [HSP(SDM1)]
At the end of the interval [symbol] t following the first SDM treatment: [HSP( [symbol] t)]
Immediately after the second SDM treatment at [symbol] t: [HSP(SDM2)]
Also shown is the improvement factor over a single treatment: β=[HSP(SDM2)]/[HSP(SDM1)]

TABLE 5
HSP concentrations at the four times just described in the text: Effect
of varying the SDM Ω for two SDM applications on a cell when
the treatments are separated by [symbol] t = 0.25 minutes = 15 seconds.

| | [HSP_{(equil)}] | [HSP_{(SDM1)}] | [HSP( [symbol] t)] | [HSP_{(SDM2)}] | β |
|---|---|---|---|---|---|
| Ω = 0.2 | 0.315 | 0.67 | 0.54 | 0.95 | 1.27 |
| Ω = 0.5 | 0.315 | 1.10 | 0.77 | 1.34 | 1.22 |
| Ω = 1.0 | 0.315 | 1.57 | 0.93 | 1.71 | 1.09 |

Table 6 is the same as Table 5, except that it is for an interval between SDM treatments of [symbol] t=0.5 minutes=30 seconds.

TABLE 6
HSP concentrations at the four times described in the text: Effect
of varying the SDM Ω for two SDM treatments on a cell when
the treatments are separated by [symbol] t = 0.5 minutes = 30 seconds.

| | [HSP_{(equil)}] | [HSP_{(SDM1)}] | [HSP( [symbol] t)] | [HSP_{(SDM2)}] | β |
|---|---|---|---|---|---|
| Ω = 0.2 | 0.315 | 0.67 | 0.44 | 0.77 | 1.14 |
| Ω = 0.5 | 0.315 | 1.10 | 0.58 | 1.18 | 1.08 |
| Ω = 1.0 | 0.315 | 1.57 | 0.67 | 1.59 | 1.01 |

Table 7 is the same as the Tables 5 and 6, except that the treatments are separated by one minute, or sixty seconds.

TABLE 7
HSP concentrations at the four times just described in
the text: Effect of varying the SDM Ω for two
SDM treatments on a normal (healthy) cell when the treatments
are separated by [symbol] t = 1 minute = 60 seconds.

| | [HSP_{(equil)}] | [HSP_{(SDM1)}] | [HSP( [symbol] t)] | [HSP_{(SDM2)}] | β |
|---|---|---|---|---|---|
| Ω = 0.2 | 0.315 | 0.67 | 0.30 | 0.64 | 0.95 |
| Ω = 0.5 | 0.315 | 1.10 | 0.37 | 1.06 | 0.96 |
| Ω = 1.0 | 0.315 | 1.57 | 0.48 | 1.51 | 0.96 |

Tables 5-7 show that:
The first treatment of SDM increases [HSP] by a large factor for all three Ω's, although the increase is larger the larger Ω. Although not displayed explicitly in the tables, the increase in [HSP] comes at the expense of the cytoplasmic reservoir of sequestered (unactivated) HSP's: [HSPHSF(SDM1)] is much smaller than [HSPHSF(equil)]
[HSP] decreases appreciably in the interval [symbol] t between the two SDM treatments, with the decrease being larger the larger [symbol] t is. (The decrease in [HSP] is accompanied by an increase in both [HSPHSF]—as shown in FIG. 44 and in [HSPS] during the interval [symbol] t—indicating a rapid replenishment of the cytoplasmic reservoir of unactivated HSP's and a rapid attachment of HSP's to the damaged proteins.)
For [symbol] t less than 60 seconds, there is an improvement in the number of activated (free) HSP's in the cytoplasm for two SDM treatments rather than a single treatment.
The improvement increases as [symbol] t becomes smaller.
For [symbol] t becoming as large as 60 seconds, however, the ratio β=[HSP(SDM2)]/[HSP(SDM1)] becomes less than unity, indicating no improvement in two SDM treatments compared to a single SDM treatment although this result can vary depending on energy source parameters and tissue type that is treated.
The improvement for [symbol] t<60 seconds is larger the smaller the SDM Arrhenius integral Ω is.

The results for the improvement ratio β=[HSP(SDM2)]/[HSP(SDM1)] are summarized in FIG. 45, where the improvement ratio β=[HSP(SDM2)]/[HSP(SDM1)] vs. interval between SDM treatments [symbol] t (in seconds) for three values of the SDM Arrhenius integral Ω, and for the three values of the interval [symbol] t=15 sec, 30 sec, and 60 sec. The uppermost curve is for Ω=0.2; the middle curve is for Ω=0.5; and the bottom curve is for Ω=1.0. These results are for the Rybinski et al (2013) rate constants of Table 3 and the equilibrium species concentrations of Table 4.

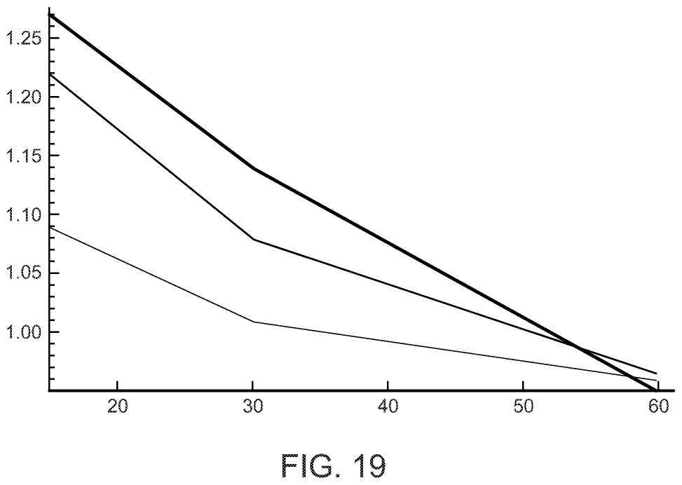

It should be appreciated that results of Tables 5-7 and FIG. 19 are for the Rybinski et al. (2013) rate constants of Table 3 and the equilibrium concentrations of Table 4. The actual concentrations and rate constants in a cell may differ from these values, and thus the number results in Tables 5-7 and FIG. 19 should be taken as representative rather than absolute. However, they are not anticipated to be significantly different. Thus, performing multiple intra-sessional treatments on a single target tissue location or area, such as a single retinal locus, with the second and subsequent treatments following the first after an interval anywhere from three seconds to three minutes, and preferably ten seconds to ninety seconds, should increase the activation of HSPs and related components and thus the efficacy of the overall treatment of the target tissue. The resulting “stair-stepping” effect achieves incremental increases in the number of heat shock proteins that are activated, enhancing the therapeutic effect of the treatment. However, if the interval of time between the first and subsequent treatments is too great, then the “stair-stepping” effect is lessened or not achieved.

The technique of the present invention is especially useful when the treatment parameters or tissue characteristics are such that the associated Arrhenius integral for activation is low, and when the interval between repeated applications is small, such as less than ninety seconds, and preferably less than a minute. Accordingly, such multiple treatments must be performed within the same treatment session, such as in a single office visit, where distinct treatments can have a window of interval of time between them so as to achieve the benefits of the technique of the present invention.

Because SDM normalizes retinal health and function by correcting RPE protein misfolding in the earlies stages of dysfunction, progression to inflammaging due to normal aging or, in more advanced dysfunction, frank neurodegenerative disease, and its consequences can be effectively slowed or prevented by early and maintained periodic treatment.

Retinal laser treatment sublethal to the RPE, such as subthreshold diode micropulse laser (SDM) acts by activating and enhancing the reaction kinetics of heat-shock protein (HSP) mediated protein repair in dysfunctional RPE cells. As a catalytic process initiating reparative cascades locally, in the retina and eye, and systemically, SDM normalizes a myriad of processes, which includes improvement in mitochondrial function, and local and systemic immunomodulation. By repairing the RPE, RPE and thus retinal function is improved and normalized physiologically. Because such SDM elicited repairs include the transcriptional and translational mechanisms within the mitochondria and cell nucleus, the effects of SDM may be broad and long-lasting. Clinical and experimental data on SDM for chronic progressive retinopathies is long and extensive.

The chemical mediators elaborated by the RPE that maintain retinal health and function act locally but are diffusible. Animal and human clinical studies have shown that these chemicals can be found in the vitreous body and aqueous humor of the eye after SDM treatment of the RPE. Without exception, the effects of SDM, and thus the function of the chemical mediators released from the RPE in response to RPE SDM HSP activation are reparative and restorative to retinal function. Further, laboratory studies show that SDM of the RPE produces local and systemic responses, including local stem cell activation and ocular recruitment of pluripotent stem cells from the bone-marrow. In one such study, SDM treatment of one eye in mice resulted in recruitment of bone-marrow derived (BMD) immune and stem cells to the retina of both eyes.

Thus, the present invention can utilize subthreshold diode micropulse laser (SDM), as illustrated and described above, to address many issues associated with normal aging and disease associated pathologies located outside the retina itself, including normal age-related reductions in visual function, normal cataract development and progression, presbyopia or age-related nearsightedness, and intraocular pressure elevations pre-disposing to and complicating open-angle glaucoma (OAG).

Normal aging is associated with subtle decreases in visual function, particularly impaired dark adaptation and mesopic (room-light) visual acuity. More severe compromises in these functions are predictive of future diseases including AMD, DR, and OAG. Because normal aging is associated with increased protein misfolding and slowing protein repair kinetics, SDM can improve subpathological age-related losses of macular, and thus central visual function. This dysfunction may be due to any or a combination of different factors. Slower metabolic activity in the RPE may be improved by SDM normalization of cell and RPE mitochondrial function. Decline in function of the neurosensory retina and optic nerve may be improved by the neurotrophic/neuroprotective effects of SDM.

Cataract formation is the most common cause of reversible age-related visual loss. As cataract is a virtually universal accompaniment to aging, it is a normal process and not a disease state. Cataract formation is the result of chemical changes in the crystalline lens that lead to disorganization, cross-linking and aggregation of lens proteins often due to oxidative changes that cause the lens to be opaque. The lens, having no blood vessels as a source of nutrition, relies entirely on the chemical environment of the aqueous fluid that surrounds it for its nutrition and health. Chemical mediators elaborated in the RPE are present in the aqueous and become abnormal in age and disease and can be normalized and improved by SDM treatment of the retinal macula. By improving and normalizing the microenvironment of the crystalline lens cataract development may be delayed, and progression slowed.

Presbyopia, or “old eyes” is a normal aging event typically coming on at approximately age 35. In presbyopia, the eye loses its ability to focus at near. Thus, the ability to read is impaired. This is generally addressed by use of reading glasses or bifocals to provide the ability to focus up close. The cause of presbyopia is one or a combination of loss of elasticity/flexibility of the crystalline lens; and loss of the ability of the ciliary muscle to contract to allow the lens to change shape as required for near vision. SDM treatment of the retina may delay the onset and/or reduce the severity of presbyopia by improving the health and function of both the crystalline lens and ciliary muscle via normalization of RPE derived chemical mediators that maintain ocular health and function.

Elevation of intraocular pressure (IOP) is a common phenomenon of aging. When severe, it may cause or contribute to optic nerve damage as open angle glaucoma. Avoiding normal age-related elevations in IOP may thus prevent sight-threatening OAG. IOP the result of two primary interactions: the rate of production of aqueous fluid by the ciliary body epithelium (essentially continuation of the RPE beyond the retina in the anterior part of the eye); and the rate of aqueous fluid egress from the eye through the epithelium of the trabecular meshwork. Imbalance may lead to high IOP. Increased resistance to flow in the trabecular meshwork is generally considered the most common abnormality, due to build-up up abnormal material blocking the meshwork. As RPE-derived chemical mediators of normal ocular function are present and circulate in the aqueous fluid, normalization via SDM treatment of the retinal macula may contribute to normalization of ciliary body aqueous production and trabecular meshwork function, reducing normal age-related IOP elevations and thus the likelihood of frank disease in OAG.

The effects of thermal laser effects sublethal to the RPE are multivalent, catalytic, reparative, restorative and functionally normalizing to the retina. Such a response is described as a physiologic “reset” phenomenon”, as it is largely agnostic to the underlying cause of retinal dysfunction. As AMD is a neurodegenerative disorder, such laser effects are also by definition neuroprotective. These include down-regulation of VEGF and up-regulation of pigment epithelial derived factor; RPE heat-shock protein (HSP) activation and acceleration HSP-mediated protein repair in unhealthy cells; improved mitochondrial function; inhibition of apoptosis; reduced indicators of degenerative chronic and increased indicators of reparative acute inflammation; decreased reactive oxygen species and increased nitrous oxide and superoxide dismutase; improved Mueller cell function; reparative local and systemic immunomodulation and stem cell activation; modulation of tissue matrix metalloproteinases; and normalized RPE cytokine, chemokine and interleukin expression and response, and improved retinal autoregulation. Thus, it is clear that SDM treatment of the retina, specifically of the macula, produces effects that improve the condition of the entire eye, not just the area of local retinal SDM treatment.

Analysis of SDM effects on the kinetics of HSP activation in the RPE shows that activation is a threshold phenomenon and thus form of bioactivation. Laser-induced increases in free intracellular HSPs are modest, consistent with the findings of in vivo and in vitro studies demonstrating low levels of HSP activation at exposure levels sublethal to the RPE. This finding is also consistent with long clinical experience and prior studies finding no notable differences in therapeutic effectiveness of retinal laser treatments based on laser wavelength. Analysis further shows that the principal laser-triggered HSP effect is not to increase the level of free and activated intracellular HSPs in the short-term (immediate, or subsecond time frame); but, rather in the longer term (minutes, hours) to induce a conformational change in free HSPs that substantially increases the rate of protein repair (via rate constant k10) specifically in sick cells characterized by high levels of damaged proteins and shortened half-lives of normal proteins. This would account for the normalization of RPE function, and thence retinal function, observed following SDM in various clinical settings independent of the cause of dysfunction (the reset phenomenon); and account for the property of pathoselectivity characteristic of SDM and other low-power laser treatments, wherein exposure improves and normalizes the function of only dysfunctional cells without any notable effect on healthy cells.

The key determinant of treatment safety for any intervention is the therapeutic range (TR). The TR of modern retinal laser therapy, extending from the first biologic effect to the 50% risk of RPE death, can be thought of as describing the “target size” of treatment. Within this range, treatment is sublethal to the RPE and thus maximized with respect to safety and efficacy, permitting amplification via high-density application to maximize therapeutic benefits.

As a rule, the TR for nanosecond lasers is zero, as they are inherently photodisruptive to the RPE (thus destroying the cell before achieving HSP activation); narrow for continuous wave (CW) lasers; and wide for micropulsed lasers, with the width or target size of the TR increasing exponentially with decreasing pulse duty cycle. For CW lasers, such as the Pattern Scanning Laser (PASCAL; Topcon, Tokyo, Japan), the TR is 0.010 watts (99.94% narrower than the TR for 810 nm SDM at a 5% duty cycle) making it theoretically possible, but clinically unlikely, to consistently “hit” within the treatment target window; treating below the TR being ineffectual, and above the TR resulting in retinal damage. Thus, the inherent unpredictability renders CW lasers unsuitable for modern retinal laser therapy, for which reliable safety is a prerequisite.

Scaling law analysis comparing the 577 nm and 810 nm parameter subthreshold micropulsed laser sets finds the TR of 577 nm 86% narrower than for 810 nm (0.23 vs 1.62 watts). This may be attributed to the observation that RPE melanin absorption of 577 nm is roughly 4× that of 810 nm. Thus, compared to 810 nm, shorter laser wavelengths such as 577 nm carry an inherently greater risk of inadvertent retinal damage, such as might result from a faulty titration algorithm, incorrect laser setting, individual patient or local retinal variations in RPE melanin density or heterogeneity, media absorption, or scatter.

This data indicates that the preferred laser parameters for panmacular SDM to reduce the effects of normal aging on visual acuity, intraocular pressure elevation, presbyopia, and cataract with maximum safety and effectiveness include use of a low pulse frequency (duty cycle of 5% or less); a long wavelength, such as between 532 nm and 1300 nm, and more preferably between 750 nm and 1000 nm, and even more preferably 810 nm in the near infrared range; combination of power, spot size and spot duration to achieve a large therapeutic range. Such variations are illustrated in Table 8 below:

TABLE 8
Therapeutic range comparison of various retinal laser modes.

| | Wave- | Retinal | | Duty | Spot | Therapeutic |
|---|---|---|---|---|---|---|
| Lase | length | Spot | Power | Cycle | Duration | Range8 |
| Mode | (nm) | (um) | (Watts) | (%) | (seconds) | (Watts) |
| Nano 1 | 532 | 400 | ~24(mJ) 4 | CW | 3 × 10−9 | 0 |
| Micro 2 | 532 | 100 | ~0.117 4 | CW | 0.00002 | 0.010 |
| MP 3, 5 | 577 | 105 | 0.250 5 | 0.20 | 0.20 | |
| MP 3,6 | 810 | 210 | 1.4 5 | 0.15 | 4 | |
| MP 3,7 | 810 | 525 | 1.7 5 | 0.30 | 15 | |

1Nanosecond continuous wave (CW).
2Microsecond CW.
3 Micropulsed.
4 Estimate, by titration.
5 Fixed, Vujosevic, et al.
6 Fixed, Luttrull, et al.
7 Fixed, currently preferred.
8 Calculated difference between the laser power at given laser parameters required for reaching the activation threshold of 1.0 for the Arrhenius integrals of the therapeutic reset effect (lower limit of TR) and the 50/50 risk of thermal cell death (upper limit of TR).

The reason that treatment of only a small posterior part of the retina, the macula, has such profound effects is that the macula is the most metabolically active part of the body, with the greatest per-weight glucose and oxygen utilization. Of the nerve fibers that arise from the retinal photoreceptors to form the optic nerve that transmits visual information to the brain, 95% originate in the macula. Reflecting this, the density/concentration of RPE cells in the macula is the highest of anywhere in the retina. As a result, virtually all useful, sharp, and color vision derives from the macula. There is little vision outside the macula. Thus, improvement in macular function, such as that elicited by SDM treatment, improves all indices of useful vision function. Further, because of the unique anatomy and activity of the macula, alteration of macular function has an outsized effect on ocular physiology and health as the most important source of the many RPE—derived chemical factors essential to normal ocular health. Because these diffuse into the ocular fluids and circulate throughout the eye they can influence the health and function of ocular tissues outside the macula including the lens, ciliary epithelium, and trabecular meshwork.

The RPE is essential to normal formation of all parts of the eye via induction. This inductive effect is carried forward into maturity as a trophic effect; that the RPE-elaborated chemical mediators are diffusible throughout the eye and that the RPE role in induction and trophism suggest a normal role of these RPE-derived chemical mediators in governing ocular health beyond the retina to those structures exposure to these mediators. RPE function and thus the makeup of these diffusible mediators and immunostimulators becomes senilic in normal aging and pathologically abnormal in disease. Abnormality of the RPE and thus RPE elaborated chemical mediators and their effects on ocular function in general can be improved by SDM treatment of the macula.

The basic SDM treatment technique is the same for all retinal treatment indications including the normal aging changes. This treatment is called “Panmacular SDM”. Preferably, the retina located between the macular retinal vascular arcades is treated confluently with the laser, including the macula and macular center, the fovea. The number of spot applications required depends on the laser spot size employed. If desired, based on the treatment indication, treatment can be extended to include the entire retina.

SDM, in accordance with the present invention, can be used in connection with other treatments which may have different mechanisms of action and may possibly have a synergistic effect with appropriate combination with SDM. For example, photobiomodulation has also been shown to improve retinal and visual function.

Photobiomodulation (PBM) employs application of high but physiologic intensity visible light to the retina. There is no thermal effect. The effects are both wavelength-specific, and order-of-presentation specific. Red appears to be the most useful and beneficial. PBM with red light to the retina may improve visual acuity and visual function in both normal older individuals, as well as those with AMD and DR. PBM acts via photoelectric effects on the respiratory chain metal cations in RPE mitochondria to improve retinal energy production and utilization. Thus far, clinical data on the benefits of PBM for retinal diseases is currently limited. However, SDM in combination with PBM, through appropriate combinations, may have a synergistic effect or different mechanisms of action in treating eyes having disorders associated with normal aging processes or other visual disorders.

Although several embodiments have been described in detail for purposes of illustration, various modifications may be made without departing from the scope and spirit of the invention. Accordingly, the invention is not to be limited, except as by the appended claims.

What is claimed is:

1. A process for photostimulating a normally aging eye, comprising the steps of:
providing a pulsed light beam having parameters of wavelength, duty cycle, power and pulse train duration selected so as to raise an eye tissue temperature to achieve a therapeutic or prophylactic effect, wherein an average temperature rise of the eye tissue over several minutes is maintained at or below a predetermined level so as to not permanently damage the eye tissue; and
applying the pulsed light beam to target tissue comprising retinal tissue of the eye for less than one second to photostimulate the eye tissue without permanently damaging the eye tissue;
wherein ocular function and health is improved so that disorders associated with a normally aging eye are slowed or prevented.

2. The process of claim 1, wherein the pulsed light beam has a wavelength between 530 nm and 1300 nm, a duty cycle of less than 10%, and a pulse train duration of between 0.1 and 0.6 seconds.

3. The process of claim 2, wherein the wavelength is between 750 nm and 1000 nm and the duty cycle is between 2% and 5%.

4. The process of claim 2, wherein the pulsed light beam has a power of between 0.5 and 74 watts.

5. The process of claim 1, wherein the applying step includes the step of raising the target tissue between six and eleven degrees Celsius at least during application of the pulsed light beam while maintaining an average target tissue temperature over several minutes below a predetermined level.

6. The process of claim 5, wherein the average target tissue temperature is maintained at one degree Celsius over a six minute period of time.

7. The process of claim 1, wherein the pulsed light beam is applied over substantially the entire retina, including at least a portion of the fovea.

8. The process of claim 1, wherein a plurality of pulsed light beams are simultaneously applied to the target tissue.

9. The process of claim 8, wherein at least a plurality of the pulsed light beams are of different wavelengths.

10. The process of claim 1, wherein the pulsed light beam is applied to a first target tissue area and between pulses of the pulsed light beam moved to one or more additional target tissue areas and then within the period of time between pulses according to the duty cycle, comprising less than one second, returned and reapplied to the first target tissue.

11. The process of claim 1, including the steps of:
performing a first treatment to a target tissue by repeatedly applying a pulsed energy to the target tissue over a first period of time comprising less than one second so as to controllably raise a temperature of the target tissue to therapeutically treat the target tissue without destroying or damaging the target tissue and to create a first level of heat shock protein activation in the target tissue;
halting the application of the pulsed energy to the target tissue for an interval of time comprising 3 seconds to 3 minutes; and
performing a second treatment to the target tissue, that received the first treatment, immediately after the interval of time by repeatedly reapplying the pulsed energy to the target tissue over a second period of time comprising less than one second so as to controllably raise the temperature of the target tissue to therapeutically treat the target tissue without destroying or damaging the target tissue and to create a second level of heat shock protein activation in the target tissue that is greater than the first level.

12. A system for photostimulating eye tissue of a normally aging eye, comprising:
at least one laser console generating at least one micropulsed treatment laser light beam, the at least one treatment laser light beam having parameters to treat the retinal tissue without damaging or destroying the retinal tissue, including having a wavelength between 750 nm and 1300 nm, a duty cycle of less than 10%, and pulse train duration of between 0.1 and 0.6 seconds;
at least one optical lens or mask that the at least one treatment laser light beam passes through to optically shape the at least one treatment laser light beam;
a coaxial wide-field non-contact digital optical viewing camera projecting the at least one treatment laser light beam to an area of a desired site of the normally aging eye for performing retinal phototherapy or photostimulation and improve ocular health and slow or prevent normally aging disorders; and
a mechanism that controllably moves the at least one treatment laser light beam over substantially the entire retina, including at least a portion of the fovea.

13. The system of claim 12, wherein the at least one laser console comprises a plurality of laser consoles.

14. The system of claim 13, wherein at least a plurality of the generated treatment laser light beams have different wavelengths.

15. The system of claim 12, wherein the mechanism controllably moves the at least one laser light beam during an interval between consecutive pulse applications of the at least one treatment laser light beam to a first treatment area to at least one other area of the desired site for performing retinal phototherapy or photostimulation, and subsequently returns the at least one treatment laser light beam to the first treatment area within a predetermined period of time comprising 1 to 3 milliseconds to apply another pulse application of the at least one treatment laser light beam to that first treatment area.

16. The system of claim 12, wherein the at least one optical lens or mask includes diffractive optics to generate a plurality of treatment light beams from the at least one treatment laser light beam which are simultaneously projected onto the retinal tissue.

17. The system of claim 12, wherein the tissue is raised between six and eleven degrees Celsius at least during application of the at least one treatment pulsed light beam while maintaining an average target tissue temperature over a six minute period below at or below one degree Celsius.
